(19) United States
(12) Patent Application Publication
(10) Pub. No.: US 2025/0144391 A1
(43) Pub. Date: May 8, 2025

(54) TAVI PRE-ASSEMBLED LOADING TOOL AND PROCEDURE

(71) Applicant: BIOTRONIK AG, Buelach (CH)

(72) Inventors: Christopher Lenherr, Winterthur (CH); Markus Hepke, Waltrop (DE); Johannes Schampel, Birmensdorf (CH); Moritz Oeler, Zuerich (CH)

(21) Appl. No.: 18/727,844

(22) PCT Filed: Feb. 10, 2023
(86) PCT No.: PCT/EP2023/053372

§ 371 Date: Jul. 10, 2024

(30) Foreign Application Priority Data
Feb. 15, 2022 (EP) 22156699.5

(51) Int. Cl. A61M 39/12; A61F 2/24; A61F 2/95

(52) U.S. Cl. CPC A61M 39/12; A61F 2/243; A61F 2/95

(57) ABSTRACT

A connector for connecting a medical implant to a delivery catheter includes a proximal portion and a distal portion connected to the proximal portion via a central portion. The proximal, distal and central portions extend along a central axis (A). A distal end of the distal portion facing away from the central portion. At least one recess is formed in the central portion and in the distal portion. The at least one recess is configured to receive a connecting element of the medical implant configured to connect the medical implant to the connector. The distal portion tapers towards the distal end. The distal portion can include a conical section, and an outer surface of the conical section includes a generatrix that encloses an acute angle (α) with the central axis (A), with the acute angle (α) in the range of between 20° and 60°.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2023/050840, which was filed Jan. 16, 2023, which application claimed priority from EP Application Serial No. 22151791.5, which was filed Jan. 17, 2022.

### FIELD OF THE INVENTION

The present invention relates to a system for mounting a medical implant, particularly an aortic heart valve prosthesis, to a delivery catheter configured for delivering the medical implant to an implantation site.

### BACKGROUND

Such a delivery catheter can include a capsule for receiving medical the implant when the latter is in a collapsed state; e.g., a crimped aortic heart valve prosthesis. The capsule is configured to cover the medical implant when the latter is arranged on a support element connected to an inner sheath of the delivery catheter while the capsule is connected to an outer sheath of the delivery catheter. Retraction of the outer sheath with respect to the inner sheath allows displacing the capsule with respect to the inner sheath and the respective support element so as to deploy and release the medical implant. With such a delivery catheter, a medical implant like an aortic heart valve prosthesis can be, e.g., only partially released and can then be retracted into the capsule for the purpose of re-positioning the medical implant so that it can be deployed at a proper implantation site in a next attempt. Reinserting the medical implant into the capsule is commonly termed “resheathing” and is for example achievable by providing a connection of the medical implant to a connector of the delivery catheter connected to the inner sheath of the delivery catheter. However, such a resheathing procedure is only possible up to a certain point of no return. Once this point has been passed upon deploying the medical implant, the latter will be completely deployed and released from the delivery catheter and can no longer be retrieved by the delivery catheter. Delivery catheters of the afore-mentioned kind, particularly for implantation of a self-expanding aortic heart valve prosthesis via a transcatheter aortic valve implantation (TAVI, also denoted as TAVR for transcatheter aortic valve replacement), are challenging in multiple aspects. A typical difficulty is to prepare such delivery catheter for an implantation of the self-expandable medical implant, particularly to mount the medical implant to the delivery catheter.

US 2013/0116770 A1 describes a system and a method for sheathing an implantable device. EP 2 865 355 B1 discloses a system for attaching an implant to a catheter shaft including at least two components which each at least partially enclose a rotationally symmetrical space tapering along the axis of symmetry. Furthermore, U.S. Pat. No. 7,743,918 B2 discloses a package for a catheter that holds the distal part of the catheter in a natural state and the proximal part of the catheter in a curled State. The package is included of two trays that are bridged by the catheter and are held together by a slidable fixing member. Further, US 2012/0305441 A1 describes a tray and packaging system for a prosthetic valve delivery system that permits a conversion from a storage and/or shipping configuration to a set up and preparation configuration. US 2017/0056149 A1 discloses a packaging system for storing a prosthetic valve and an elongated delivery system in a non-fluid environment, wherein the packaging system includes a tray that includes a cavity sized and shaped to house a valve cover containing the prosthetic valve and at least part of the distal portion of the elongated delivery system. Further, WO 2018/204455 A1 relates to wet transcatheter prosthetic heart valve packaging and assemblies in which a prosthetic heart valve is loaded into a first portion of a delivery device and positioned within a container in which sterilizing fluid is retained to sterilize interior portions of the container as well as provide moisture to prevent the implant from drying out. Finally, US 2015/0305867 A1 discloses a delivery apparatus suited for advancing a prosthetic heart valve through the aorta (i.e., in a retrograde approach) for replacing a diseased native aortic valve, wherein the delivery apparatus is configured to deploy a prosthetic valve from a delivery sheath in a precise and controlled manner at the target location within the body.

Preparing/Loading a delivery catheter of the afore-mentioned kind usually is a multi-step procedure that involves manual handling of the delivery catheter and medical implant (e.g. with forceps) to be mounted thereto and is therefore prone to errors. Therefore, based on the above, the problem to be solved by the present invention is to provide a system for mounting a medical implant, particularly a self-expandable or self-expanding aortic heart valve prosthesis (e.g. a TAVI prosthesis) or stent, to a delivery catheter configured for delivering the medical implant to an implantation site, which system allows a safe mounting of the medical implant to the delivery catheter and can be conducted by a single person.

### SUMMARY OF THE INVENTION

A preferred embodiment connector for connecting a medical implant to a delivery catheter includes a proximal portion and a distal portion connected to the proximal portion via a central portion. The proximal, distal and central portions extend along a central axis (A). A distal end of the distal portion facing away from the central portion. At least one recess is formed in the central portion and in the distal portion. The at least one recess is configured to receive a connecting element of the medical implant configured to connect the medical implant to the connector. The distal portion tapers towards the distal end. Preferably, the distal portion includes a conical section, and an outer surface of the conical section includes a generatrix that encloses an acute angle (α) with the central axis (A), and the acute angle (α) is in the range of between 20° and 60°.

A preferred system includes a connector as described above and the system is configured for connecting a medical implant to a delivery catheter configured for delivering the medical implant to an implantation site. The system includes the medical implant in the form of a self-expandable or self-expanding scaffold. A delivery catheter is configured to deliver the medical implant to an implantation site. The delivery catheter includes a capsule for covering the medical implant and at least one connector. A loading cone is configured to arrange a proximal end portion of the scaffold in a crimped state by decreasing an outer diameter of the proximal end portion of the scaffold, wherein the loading cone includes a body with a distal opening configured to receive the scaffold and an opposing proximal opening. The distal opening has a diameter that is larger than a diameter of the proximal opening. The body of the loading cone has an inner surface extending from the distal opening to the proximal opening. The inner surface tapers towards the proximal opening. A loading tube is configured to be inserted into the proximal opening of the loading cone and to enclose the capsule of the delivery catheter. The loading cone and the loading tube are pre-mounted on the delivery catheter, and packaging encloses the delivery catheter, the loading cone, and the loading tube pre-mounted on the delivery catheter.

### BRIEF DESCRIPTION OF THE DRAWINGS

In the following, further features, advantages and embodiments of the present invention are explained with reference to the Figures, wherein

FIG. 1 shows a schematic top view onto an embodiment of a system according to the present invention,

FIG. 2 shows a flow chart of an embodiment of a method for mounting a medical implant, here in form of an aortic heart valve prosthesis configured for TAVI, to a delivery catheter,

FIG. 3 shows a perspective view of a system for mounting the medical implant to the delivery catheter, wherein the packaging is arranged in an initial elongated state,

FIG. 4 shows a perspective view of the system shown in FIG. 3 in an operating state,

FIG. 5 shows the system of FIG. 4, wherein the three rinsing basins and the loading basin for holding the distal portion of the delivery catheter are filled with a saline solution for rinsing the medical implant and the delivery catheter,

FIG. 6 shows a perspective view of an implant storage jar with an implant connected to a carrier and an implant storage jar lid and a splay tube in a not assembled state;

FIG. 7 shows a perspective view of the implant storage jar of FIG. 6 with the carrier and medical implant therein, wherein the splay tube device is connected to the carrier;

FIG. 8 shows a perspective view of a rinsing process of the medical implant connected to the carrier using the three rinsing basins filled with saline solution;

FIGS. 9-10 show perspective views of the process of passing a nose cone of the delivery catheter into the splay tube of the splay tube device in order to insert the medical implant into the loading cone arranged on the delivery catheter for crimping a proximal end portion of the scaffold of the medical implant;

FIGS. 11-13 show perspective views of the splay tube device, loading cone and loading tube upon engaging of the connecting elements of the scaffold of the medical implant with the recesses of a connector of the delivery catheter;

FIGS. 14-15 show securing the connecting elements of the scaffold in the recesses of the connector with the loading tube;

FIG. 16A shows a perspective view of the medical implant connected to the carrier and inserted into the loading cone, wherein the connecting elements are connected to the connector and maintained in the engaged state by the loading tube that encloses the capsule and connecting elements;

FIG. 16B shows the loading tube in a non-engaged state being pulled out of the loading cone and both the loading tube and the loading cone are removed from the delivery catheter;

FIG. 17 shows a cross sectional view of a splay tube device of an embodiment of the system according to the present invention;

FIG. 18 shows an alternative embodiment of the splay tube device;

FIG. 19 shows a perspective view of a loading cone according to an embodiment of the present invention;

FIG. 20 shows a cross-sectional view of the loading cone of FIG. 19;

FIGS. 21-24 shows the process of inserting the medical implant into the loading cone so that it slides along the inner surface of the loading cone for crimping a proximal end portion of the scaffold of the medical implant;

FIG. 25 shows a cross sectional view of the loading cone of FIG. 19, wherein the posts of a carrier of the medical implant are guided by slots of the loading cone;

FIGS. 26-27 show perspective views of an embodiment of a carrier of a system according to the present invention, without medical implant (FIG. 26) and with medical implant pre-mounted to the carrier (FIG. 27);

FIG. 28 shows a perspective view of a base plate of the carrier shown in FIGS. 26 to 27;

FIG. 29 shows a lateral view of an embodiment of a jar of a system according to the present invention, wherein the medical implant is arranged in the jar together with the carrier shown in FIGS. 26 to 28;

FIG. 30 shows a perspective view of a detail of the carrier shown in FIG. 27;

FIG. 31 shows a top view onto the base plate of the carrier shown in FIG. 30;

FIGS. 32-33 show a connection between the splay tube device and the base plate of the carrier shown in FIGS. 30 to 31;

FIG. 34 shows a perspective view of a detail of the base plate of the carrier shown in FIGS. 32 to 33;

FIG. 35 shows a perspective view of a detail of the mounting plate shown in FIG. 31;

FIG. 36 shows a cross sectional view of a connection between the base plate of the carrier and the loading cone;

FIGS. 37-39 show details of the base plate of FIG. 31 with respect to the connections between the base plate and the posts of the carrier;

FIGS. 40-44 indicate the process of connecting the posts of the carrier to the base plate of the carrier;

FIG. 45 shows a lateral view of the medical implant pre-mounted to the carrier;

FIG. 46 shows a detail of the connection between the medical implant and the posts of the carrier;

FIG. 47 shows a detail of an alternative connection between the medical implant and the posts of the carrier;

FIG. 48 shows a cross sectional view of a post of the carrier upon insertion into a corresponding slot of the loading cone;

FIG. 49 shows a top view onto the carrier and medical implant shown in FIG. 45;

FIGS. 50-52 show a tool for controlled bending of the posts of the carrier;

FIG. 53 shows an embodiment of fixing the loading cone and the carrier and splay tube device connected thereto with respect to the tray of the packaging of the delivery catheter;

FIGS. 54-56 show insertion of the loading tube into the proximal opening of the body of the loading cone;

FIG. 57 show a cross sectional view of a loading tube and loading cone;

FIG. 58 show a side view of the loading cone;

FIG. 59A show a cross sectional view of the loading tube;

FIG. 59B show a bottom view of the loading tube;

FIGS. 60-61 show embodiments of a sloped connector of an embodiment of the system/delivery catheter according to the present invention;

FIG. 62 shows a distal portion of a delivery catheter of an embodiment of a system according to the present invention, which delivery catheter includes a sloped connector according to FIG. 60 or FIG. 61;

FIGS. 63-67 show a portion of a prosthesis in a collapsed state and a detail of a delivery catheter, particularly the connector.

### DETAILED DESCRIPTION OF THE PREFERRED EMBODIMENTS

A system for mounting a medical implant to a delivery catheter configured for delivering the medical implant to an implantation site, wherein the system includes:
a delivery catheter configured to deliver the medical implant to an implantation site, wherein the delivery catheter includes a capsule for covering the medical implant;
a loading cone for arranging a medical implant, wherein the loading cone includes a body including a distal opening for receiving the medical implant and an opposing proximal opening, wherein the distal opening includes a diameter that is larger than a diameter of the proximal opening, and wherein the body of the loading cone further includes an inner surface extending from the distal opening to the proximal opening, wherein the inner surface tapers towards the proximal opening;
a loading tube configured to be inserted into the proximal opening of the loading cone and to enclose the capsule of the delivery catheter;
wherein the loading cone and the loading tube are pre-mounted on the delivery catheter, and wherein the system includes a packaging enclosing the delivery catheter as well as the loading cone and the loading tube pre-mounted on the delivery catheter
and optionally a medical implant, preferably wherein the medical implant includes a self-expandable or self-expanding scaffold.

Also disclosed is a system for mounting a medical implant to a delivery catheter configured for delivering the medical implant to an implantation site is disclosed, wherein the system includes:
a medical implant, wherein the medical implant includes a self-expandable scaffold;
a delivery catheter configured to deliver the medical implant to an implantation site, wherein the delivery catheter includes a capsule for covering the medical implant;
a loading cone for arranging an end portion of the scaffold in a crimped state by decreasing an outer diameter of the end portion of the scaffold, wherein the loading cone includes a body including a distal opening for receiving a scaffold and an opposing proximal opening, wherein the distal opening includes a diameter that is larger than a diameter of the proximal opening, and wherein the body of the loading cone further includes an inner surface extending from the distal opening to the proximal opening, wherein the inner surface tapers towards the proximal opening so that the proximal end portion of the scaffold transitions to the crimped state when the scaffold is inserted through the distal opening into the loading cone to slide along the inner surface towards the proximal opening;
a loading tube configured to be inserted into the proximal opening of the loading cone and to enclose the capsule of the delivery catheter; and wherein the loading cone and the loading tube are pre-mounted on the delivery catheter, and wherein the system includes a packaging enclosing the delivering catheter as well as the loading cone and the loading tube pre-mounted on the delivery catheter.

Preferably, the inner surface includes a conical shape or cone shape. The cone shape or conical shape enables a sliding of the medical implant along the inner surface of the loading cone towards the proximal opening. Particularly, the fact that the loading tube and the loading cone are pre-mounted on the delivery catheter means that the delivery catheter extends through the loading tube and through the proximal and distal opening of the loading cone.

Furthermore, the loading tube is configured to support a distal end of the capsule, wherein an inner diameter of the loading tube prevents the capsule from flaring and gives it radial support.

Further, particularly, the loading cone's main purposes are to crimp the outflow end portion of the scaffold and help guide it into the loading tube or capsule.

In the framework of the present invention, the notion “distal” refers to a portion or component of the system or the implant that is remote from the handle or from the physician that operates the delivery catheter as seen along the course of the delivery catheter while the notion “proximal” refers to those portions or components that are closer to the handle of the delivery catheter or closer to the physician along the course of the delivery catheter.

According to a preferred embodiment of the system, the latter includes a carrier, wherein the medical implant is pre-mounted to the carrier.

Furthermore, according to an embodiment, the system includes a jar for storing the medical implant being pre-mounted to the carrier. Particularly, the jar can include a lid for closing the jar.

According to a further embodiment of the system, the proximal end portion of the scaffold includes connecting elements, wherein the delivery catheter includes a connector, wherein each connecting element is configured to engage with a corresponding recess of the connector. Particularly, the scaffold can be formed by a plurality of interconnected struts, wherein each connecting element is connected to at least one strut of the scaffold. The scaffold can be made out of a suitable alloy, such as a nickel titanium alloy (e.g. Nitinol), so that the scaffold includes a self-expanding property, i.e., the scaffold will automatically self-expand from a crimped state to an expanded state when an outer diameter of the scaffold is no longer limited (e.g. by a capsule surrounding the crimped scaffold).

Furthermore, according to an embodiment, the loading cone is configured for receiving the medical implant pre-mounted to the carrier through the distal opening of the loading cone with the proximal end portion of the scaffold ahead so that the proximal end portion of the scaffold transitions to the crimped state upon sliding along the inner surface of the loading cone and eventually protrudes out of the proximal opening of the body of the loading cone.

Further, according to a preferred embodiment, the carrier includes a plurality of posts protruding from a base plate of the carrier, wherein the posts are configured to secure the medical implant to the carrier, and wherein the respective post is configured to be inserted into an associated slot formed in the body of the loading cone for guiding the carrier when the medical implant is being received by the loading cone.

According to a further embodiment, the medical implant is configured to be released from the posts upon sliding along the inner surface of the loading cone to protrude out of the proximal opening with the proximal end portion of the scaffold.

Preferably, in an embodiment, the carrier is configured to be connected to the body of the loading cone by latching noses configured to engage with a groove formed in the body of the loading cone.

Furthermore, according to a preferred embodiment, the system includes a splay tube device that is configured to spread the connecting elements apart from one another when the proximal end portion of the scaffold is in the crimped state and protrudes out of the proximal opening of the body of the loading cone so that each connecting element is able to engage with the corresponding recess of the connector.

According to a preferred embodiment, the splay tube device further includes a splay tube that is movably arranged with respect to a spring retainer, wherein the splay tube can be moved from a retracted position into an advanced position when the spring retainer is connected to the carrier, wherein in the advanced position the splay tube extends through the loading cone and protrudes out of the proximal opening of body the loading cone to spread the connecting elements apart from one another, wherein the spring retainer holds a spring element that is configured to be pretensioned against the splay tube when the latter is moved into the advanced position so that the spring element generates a restoring force that tries to move the splay tube back to the retracted position.

Furthermore, in an embodiment, the splay tube device forms a handle when connected to the carrier for removing the carrier and the medical implant pre-mounted thereon from the jar.

According to yet another embodiment, the connector includes a proximal portion and a distal portion connected to the proximal portion via a central portion of the connector, wherein the distal portion includes a distal end, and wherein the respective recess is formed in the central portion and in the distal portion, and wherein the distal portion tapers towards the distal end, so that the connecting elements are spread apart from one another upon sliding along the sloped distal portion of the connector towards the respective recess of the connector. Particularly, in case the system includes such a sloped connector, the splay tube device may be omitted since the connecting elements can be spread apart by the tapering distal portion of the connector.

Furthermore, according to an embodiment, the carrier includes latching noses configured to engage with a groove formed in the spring retainer or the splay tube.

According to a further embodiment of the present invention, the loading cone includes a loading tube guide connected via spacer bridges to the body of the loading cone, wherein the loading tube guide forms a guiding opening for receiving the loading tube, which guiding opening is aligned with the proximal opening of the body of the loading cone.

Preferably, the proximal opening of the body of the loading cone includes a ledge according to an embodiment, which ledge forms a stop for the loading tube.

Furthermore, the loading tube preferably includes a head and an end portion connected to the head according to an embodiment, wherein the end portion includes a ledge on an inner surface of the tube, which ledge is configured to prevent the capsule from traveling into the loading cone. Particularly, the head of the loading tube facilitates the handling of the loading tube and may form a stop for limiting a movement of the loading tube into the proximal opening of the body of the loading cone.

According to yet another embodiment, the loading tube includes (four) slots formed into an end of the loading tube, which slots allow the end to spread for letting a nose cone of the delivery catheter pass through the loading tube. When the loading tube is fully inserted into the proximal opening of the loading cone, this radial expansion is limited by the proximal opening in the loading cone, so the nose cone can only pass through the loading tube if the loading tube is not fully inserted into the loading cone.

According to yet another aspect of the present invention, a method for mounting a medical implant to a delivery catheter according to the present invention is disclosed, wherein the method includes the steps of:
inserting the medical implant pre-mounted to the carrier with the proximal end portion of the scaffold of the medical implant ahead into the loading cone through the distal opening of the loading cone so that the proximal end portion of the scaffold transitions to the crimped state upon sliding along the inner surface of the loading cone and protrudes out of the proximal opening of the body of the loading cone,
moving the splay tube into its advanced position to spread the connecting elements of the proximal end portion of the scaffold apart and aligning the connecting elements with the recesses of the connector, and moving the splay tube into the retracted position to let the connecting elements engage with the recesses of the connector,
moving the loading tube guided by the loading cone into the proximal opening of the body of the loading cone to cover the connecting elements being engaged with the recesses of the connector to prevent the connecting elements from moving out of the recesses of the connector due to the self-expanding property of the scaffold, and
moving the capsule into the loading tube to cover the medical implant and pulling the loading tube out of the proximal opening of the body of the loading cone and extracting both loading tube and loading cone from the delivery catheter.

Furthermore, yet another aspect of the present invention relates to a connector for connecting a medical implant, particularly a self-expandable prosthesis, to a delivery catheter.

As already described above, reinserting the medical implant into the capsule is commonly termed re-sheathing and is for example achievable by providing a connection of the self-expandable prosthesis to a connector connected to a distal portion of the inner sheath of the delivery catheter.

When the medical implant/prosthesis is in its collapsed state, it has to be partially spread to be connectable to the connector. In the state of the art, tools such as tubes are known that spread the prosthesis device, particularly the stent, such that it can be connected to the connector. Hence, according to state of the art, the loading process requires an additional tool, e.g. the splay tube, and involves an additional process step. This increases the number of parts needed for loading. Further, the time needed for loading is increased due to the additional process steps. Therefore, a device is of interest that simplifies the loading process, i.e. the connection of the prosthesis to the connector, in particular to the delivery catheter or to the connector of the delivery catheter.

This problem is solved by a connector having the features of claim 15. Embodiments of this aspect of the present invention are described below.

According to claim 15 a connector for connecting a self-expanding prosthesis to a delivery catheter is disclosed. The self-expandable prosthesis includes at least one connecting element, particularly a plurality of connecting elements such as two or three connecting elements. The connector extends along a central axis, wherein the connector includes a proximal portion and a distal portion connected to the proximal portion through a central portion of the connector. Alternatively, the connector can include a proximal portion and a distal portion being integrally formed. The distal portion includes a distal end facing away from the central portion. The connector includes at least one recess formed in the central portion and in the distal portion, wherein the at least one recess is configured to receive the at least one connecting element of the self-expandable prosthesis. According to the invention, the distal portion of the connector tapers towards the distal end. Particularly, the notion “distal” means, that a distal component (e.g. a portion, section etc.) of the connector is further away along the delivery catheter from a handle or a user of the delivery catheter than a corresponding “proximal” component, when the connector is connected to the delivery catheter as intended.

According to an embodiment, the connector includes a lumen that extends along the central axis. When the connector is mounted to a delivery catheter, a guide wire can extend in this lumen through the connector and towards an outlet formed in a catheter tip of the delivery catheter.

Particularly, as described above, the prosthesis (e.g. an aortic heart valve prosthesis) can include a self-expandable tubular stent that includes a plurality of interconnected struts that form a tubular cell structure surrounding an interior space of the stent in which interior space a valve member may be arranged. Blood can flow into and out of this interior space through axial openings of the stent thereby passing a valve member of the prosthesis that is arranged in said interior space and connected to the stent. Particularly, the at least one connecting element is arranged at an end of the stent and is connected to at least one strut of the stent. The connecting element can include an eyelet.

Perpendicular to the central axis, the distal portion of the connector can have a circular cross-sectional contour. A diameter of the contour can decrease towards the distal end due to said tapering. The diameter can decrease continuously.

The distal portion and the central portion can be integrally joined together (e.g. can be formed out of a metal or another suitable material, e.g. a plastic material such as a polymer). Furthermore, also the proximal portion and the central portion are preferably integrally joined together. According to an embodiment, the distal portion, the central portion and the proximal portion are integrally joined together to form a single continuous body.

According to an embodiment, the distal end of the connector is configured to be inserted into a proximal axial opening of the prosthesis, particularly of its stent, when the prosthesis, particularly the stent, is in a collapsed state such that the at least one connecting element slides on the distal portion of the connector and engages with the associated at least one recess of the connector. Particularly, the proximal axial opening is arranged between a plurality of connecting elements of the stent that are each connected to at least one strut of the stent at a proximal end of the stent.

Particularly, the notion “collapsed state” means, that the stent, particularly the prosthesis as a whole, is radially compressed to assume a smaller diameter compared to a fully expanded state of the stent/prosthesis. Since the stent is self-expandable or self-expanding, the diameter of the stent has to be delimited so as to keep the stent in the collapsed state (when loaded into the delivery catheter, the capsule of the delivery catheter can cover the stent/prosthesis and keeps it in this collapsed state). Once the delimiting feature (e.g. the capsule) is taken away, the stent expands in the radial direction (i.e. perpendicular to the central axis) and the prosthesis is deployed. Partial deployment is possible by keeping the at least one connecting element covered with the capsule when the at least one connecting element is engaged with the at least one recess, which allows re-sheathing of the prosthesis/stent.

According to an embodiment, the connector is configured to bend the at least one connecting element radially outwards when it is moved into the proximal axial opening of the stent with the distal end ahead when the stent/prosthesis assumes its collapsed state. In case of several connecting elements, the connecting elements are spread apart in the radial direction by the distal portion of the connector while sliding on the tapered distal portion, so that eventually the connecting elements fall into the respective recess of the connector which connects the prosthesis to the connector. This connection can be maintained by covering the connecting elements with the capsule so that the latter covers the connecting elements and the prosthesis/stent.

Particularly, according to an embodiment, the connector, in particular the distal portion, is configured to guide the at least one connecting element of the prosthesis towards the at least one recess of the connector, when the connector is coaxially aligned with the stent and moved towards the proximal axial opening of the stent. Particularly due to said guiding, the at least one connecting element can slide up the distal portion of the connector to fall into the at least one recess.

An advantage of the connector according to the present invention is that it fulfils two tasks, the bending of the respective connecting element of the self-expandable stent/prosthesis and the fastening of the prosthesis to the connector. This simplifies the process of loading the prosthesis to the delivery catheter that is used for delivering the prosthesis (e.g. via TAVI). No additional tools are needed for deforming the stent/prosthesis such that it can be connected to the connector. Hence, less equipment is needed to perform the loading operation, such that costs are advantageously reduced. Moreover, the time needed to load the prosthesis to the delivery catheter is decreased because the connector is configured such that bending of the connecting element(s) and anchoring occur in one combined loading step.

Furthermore, in an embodiment, the proximal portion of the connector includes a proximal end facing away from the central portion, wherein the proximal portion tapers towards the proximal end, too. Particularly, the proximal end faces away from the distal end of the connector.

Further, according to an embodiment, perpendicular to the central axis, the proximal portion of the connector includes a circular cross-sectional contour. The proximal portion can be conical or can include a proximal conical section. The proximal conical section can form the proximal end of the proximal portion of the connector.

According to an embodiment, an outer surface of the distal portion of the connector (or of the conical section of the distal portion) includes a generatrix that encloses an acute angle with the central axis of the connector, wherein in an embodiment, the acute angle may be in the range of between 20° and 60°, preferably of between 25° and 45°, more preferably of between 3° and 40°. The generatrix is a straight or curved line that, when moved along a given path, here rotated about the central axis along the circumferential direction of the connector, generates a surface corresponding to the outer surface of the distal portion (or of the conical section of the distal portion) of the connector.

Furthermore, according to an embodiment, an outer surface of the proximal portion or of the proximal conical section of the proximal portion of the connector includes a generatrix that encloses an acute angle with the central axis, wherein this acute angle is preferably in the range the range of between 20° and 60°, preferably of between 25° and 45°, more preferably of between 3° and 40°. The acute angle of the proximal portion may be the same as or different from the acute angle of the distal portion.

In a further embodiment, the at least one recess of the connector includes a groove and a pocket connected with the groove via a narrow section of the at least one recess, wherein the groove is preferably formed in the distal portion of the connector and the pocket is preferably formed in the central portion of the connector. The narrow section can be formed in an intermediate portion connecting the distal portion to the central portion. The narrow section can also be formed in the central portion and or in the distal portion. Particularly, the groove is formed in the conical section of the distal portion of the connector. Particularly, the intermediate portion can integrally connect the distal portion to the central portion and may provide a rounded transition between the distal portion and the central portion of the connector.

According to a preferred embodiment, the pocket may be configured to receive the at least one connecting element of the prosthesis. Furthermore, particularly, the groove can be configured to receive a strut of the stent to which the at least one connecting element is connected. According to an embodiment, particularly due to the narrow section, the pocket forms an undercut, so that the at least one connecting element can engage behind the undercut formed by the pocket. In case the pocket is now covered with a capsule, the at least one connecting element being engaged with the pocket/undercut cannot be released from the connector unless the capsule is pulled away from the pocket to release the connecting element. Particularly, the groove tapers towards the pocket to form said narrow section of the at least one recess.

Furthermore, according to an embodiment, the narrow section of the at least one recess includes a width extending in a circumferential direction of the connector and the pocket includes a pocket width extending in the circumferential direction of the connector, wherein the width of the narrow section is smaller than the pocket width.

In an embodiment, the at least one recess of the connector is configured such that when the prosthesis is connected to the connector, the at least one connecting element resides in the pocket, wherein the undercut is configured to anchor the connecting element to the connector. This is particularly achieved by letting the connecting element engage behind the undercut when it resides in said pocket.

Such a configuration allows an easy connection between the respective connecting element of the stent and the connector. Further, the prosthesis can be easily released from the connector, when necessary, by allowing the respective connecting element to jump out of the associated pocket (e.g. by removing a capsule holding the respective connecting element in the associated pocket).

According to a further embodiment, the distal portion of the connector includes a distal first section and a proximal second section, wherein particularly, with respect to the central axis, an outer surface of the first section include a first slope and an outer surface of the second section includes a second slope, wherein preferably the first slope is smaller than the second slope.

The force acting on the respective connecting element in the radial direction due to the distal portion of the connector can be influenced by the slope of the tapering of the distal portion. Particularly, by corresponding selection of the slope of the first section of the distal portion, the connector can be configured such that the first section can be easily inserted in the proximal axial opening of the prosthesis. Furthermore, the slope of the second section of the distal portion of the connector can be configured so as to allow bending of the connecting element(s) with a desired force.

Furthermore, in an embodiment, the first section includes a first length and the second section includes a second length along the central axis, wherein preferably the first length equals the second length.

Furthermore, particularly, the groove of the at least one recess can be formed in the second section of the distal portion of the connector. In an embodiment, the groove is formed in the first section and in the second section of the distal portion. The groove can span the entire second section or the entire first and second section.

Particularly, in an embodiment, the first section of the distal portion adjoins the second section of the distal portion of the connector. Furthermore, the second section is connected to the central portion, particularly via the intermediate portion of the connector (see also above). Further, in an embodiment, the first section forms the distal end of the distal portion of the connector. Preferably, the first section is integrally formed with the second section of the distal portion of the connector.

In a further embodiment, the first section of the distal portion of the connector is a conical section including an outer surface and the second section of the distal portion is a conical section including an outer surface, wherein the outer surface of the first section includes a generatrix that encloses a first acute angle with the central axis and the outer surface of the second section of the distal portion includes a generatrix that encloses a second acute angle with the central axis, wherein the first acute angle is smaller than the second acute angle. The two generatrixes enclose an obtuse angle. The first angle may be between 20° and 25°, preferably 21°. The second angle may be between 31° and 40°, preferably 32°.

According to an embodiment, the first section and the second section of the distal portion of the connector enclose an obtuse angle, wherein the obtuse angle is greater than 90°, particularly greater than 120°, particularly greater than 135° and preferably less than 180°.

According to a further embodiment of the present invention, the groove of the at least one recess of the connector includes a groove bottom including a first groove bottom section arranged in the first section of the distal portion and a second groove bottom section arranged in the second section of the distal portion of the connector, wherein—with respect to the central axis—a slope of the first groove bottom section is larger than a slope of the second groove bottom section.

Furthermore, according to an embodiment, the pocket of the at least one recess of the connector includes a pocket bottom. The pocket bottom can include a slope with respect to the central axis of the connector. In an embodiment, this pocket bottom slope equals the slope of the second groove bottom section. Particularly, the pocket bottom and the second groove bottom section can extend in a common plane.

Further, in an embodiment, the slope of the first groove bottom section is larger than the slope of the first section of the distal portion of the connector. According to a further embodiment, the slope of the second groove bottom section is smaller than the slope of the second section of the distal portion of the connector.

In a further embodiment, the connector includes a plurality of recesses, preferably at least two or three recesses, wherein each of these recesses can be configured like the at least one recess described above, i.e., according to at least one of the embodiments described herein with respect to the at least one recess. Particularly, each of the recesses of the plurality of recesses is configured to receive an associated connecting element of the self-expandable prosthesis, particularly an associated connecting element of the stent of the prosthesis.

According to an embodiment, the recesses are equidistantly spaced in the circumferential direction of the connector.

A further aspect of the present invention relates to a delivery catheter for implanting an implantable prosthesis such as an aortic heart valve prosthesis (see e.g. above), wherein the delivery catheter includes an inner sheath and a connector according to the present invention, wherein the connector is connected to a distal end portion of the inner sheath of the delivery catheter. According to an embodiment, the delivery catheter further includes an outer sheath and a capsule that is connected to a distal end of the outer sheath, wherein the outer sheath surrounds the inner sheath. Particularly, the outer sheath is configured to cover the prosthesis when the latter is connected to the connector, wherein the outer sheath and therewith the capsule is movable with respect to the inner sheath to withdraw the capsule from the prosthesis and to thereby release the prosthesis from the connector.

According to an embodiment of the delivery catheter, the delivery catheter can include a prosthesis that is connectable to the connector or that is connected to the connector, wherein preferably the prosthesis is an aortic heart valve prosthesis. According to an embodiment, the prosthesis includes a self-expandable tubular stent that includes a plurality of interconnected struts that form a tubular cell structure surrounding an interior space of the stent, in which interior space a valve member of the prosthesis is arranged and connected to the stent. The stent includes a proximal axial opening (through which blood can exit the prosthesis in the implanted state). Particularly, the at least one connecting element is arranged at a proximal end of the stent and is connected to at least one strut of the stent. The connecting element can include an eyelet. Preferably, the stent includes a plurality of connecting elements, wherein each connecting element is connected to at least one strut at the proximal end of the stent. The proximal axial opening is arranged between the connecting elements.

A further aspect of the invention relates to a method for connecting a self-expandable prosthesis (for example an aortic heart valve prosthesis as described herein) to a connector according to the present invention, including the steps of:
providing the self-expandable prosthesis in a (partially) collapsed state, wherein the prosthesis includes at least one connecting element, and
causing the connecting element to slide on the connector such that the connecting element engages with the at least one recess of the connector.

The self-expandable stent can be maintained in the collapsed state by any suitable means, e.g. by a circumferential member that encloses the prosthesis, particularly the stent, so as to prevent it from expanding in the radial direction.

As described above, the connector can include a plurality of recesses (e.g. three such recesses). In this case, the prosthesis, particularly the stent, includes a corresponding number of connecting elements. Here the connecting elements are caused to slide on the connector such that the respective connecting element engages with the associated recess of the connector.

Particularly, the step of letting the connecting elements slide on the connector (e.g. on the distal end portion of the connector) includes inserting the distal end of the connector into a proximal axial opening of the prosthesis in an insertion direction, thereby spreading the connecting elements apart by the distal portion of the connector, wherein the connector is inserted such into the proximal axial opening of the prosthesis that each connecting element engages with its associated recess of the connector. Particularly, said proximal axial opening of the prosthesis is formed by the stent of the prosthesis and is arranged between the connecting elements. The connecting elements are preferably formed as eyelets (see also above).

When the respective connecting element engages with its associated recess it engages behind the undercut of the pocket of the associated recess of the connector which connects the respective connecting element to the connector.

According to an embodiment of the method, the method can include the further step of aligning the respective connecting element with an associated recess of the connector by rotating the connector about the central axis.

Furthermore, according to an embodiment of the method, the method can include the further step of moving the connector in a direction opposite the insertion direction so as to achieve engagement of the respective connecting element with an associated recess, particularly in case the respective connecting element has been moved past its associated recess during initial insertion of the connector in the axial opening. This opposite movement is facilitated by the slope of the proximal portion of the connector.

In the method according to the present invention, insertion of the connector into the axial opening of the prosthesis/stent can be achieved by moving the connector and/or the prosthesis.

Due to the method according to the present invention, the prosthesis can be easily connected to the connector, particularly to the delivery catheter, without the need of additional tools to spread the prosthesis in order to connect it to the connector. This reduces the complexity of the equipment needed as well as the time needed for loading the prosthesis to the connector.

Further examples are described below:

A connector for connecting a medical implant, preferably an implantable self-expandable or self-expanding prosthesis, to a delivery catheter, wherein the connector extends along a central axis and includes a proximal portion and a distal portion connected to the proximal portion via a central portion of the connector, wherein the distal portion includes a distal end facing away from the central portion, and wherein the connector includes at least one recess formed in the central portion and in the distal portion, wherein the at least one recess is configured to receive a connecting element of the medical implant, preferably the self-expandable or self-expanding prosthesis, for connecting the medical implant, preferably the self-expandable or self-expanding prosthesis, to the connector, wherein the distal portion tapers (continuously) towards the distal end.

The proximal portion can include a proximal end facing away from the central portion, wherein the proximal portion tapers towards the proximal end.

The distal portion can include a conical section, wherein an outer surface of the conical section includes a generatrix that encloses an acute angle with the central axis, wherein the acute angle is in the range of between 20° and 60°, preferably of between 25° and 45°, more preferably of between 3° and 40°.

The at least one recess can include a groove and a pocket connected to the groove via a narrow section of the at least one recess, wherein the groove is formed in the distal portion and the pocket is formed in the central portion.

The groove can taper towards the pocket.

The narrow section can include a width extending in a circumferential direction of the connector, and the pocket can include a pocket width extending in the circumferential direction of the connector, wherein the width is smaller than the pocket width.

7The at least one recess can be configured such that the pocket forms an undercut.

The distal portion can include a distal first section and a proximal second section.

The first section can adjoin the second section.

The first section can be a conical section including an outer surface and the second section is a conical section including an outer surface, wherein the outer surface of the first section includes a generatrix that encloses a first acute angle with the central axis and the outer surface of the second section includes a generatrix that encloses a second acute angle with the central axis, wherein the first acute angle is smaller than the second acute angle.

The generatrixes of the first section and of the second section can enclose an obtuse angle, wherein the obtuse angle is larger than 100°, particularly larger than 120°, more particularly larger than 135° or larger than 150°.

The groove can include a groove bottom including a first groove bottom section arranged in the first section of the distal portion and a second groove bottom section arranged in the second section of the distal portion, wherein a slope of the first groove bottom section is larger than a slope of the second groove bottom section.

The connector can include at least two, preferably three, recesses, wherein each recess is formed in the central portion and in the distal portion, wherein each recess is configured to receive a connecting element of the medical implant, preferably the self-expandable or self-expanding prosthesis.

A delivery catheter includes a connector according to one of the preceding examples and an inner sheath including a distal end portion wherein the connector is connected to the distal end portion of the inner sheath.

A method for connecting a medical implant, preferably a self-expandable or self-expanding prosthesis, to a connector of a delivery catheter, includes the steps of:
providing the medical implant, preferably the self-expandable or self-expanding prosthesis, in a (partially) collapsed state, wherein the medical implant, preferably the self-expandable or self-expanding prosthesis, includes at least one connecting element, preferably two or three connecting elements,
providing a connector according to one of the above examples,
letting the at least one connecting element slide on the connector such that the connecting element engages with the at least one recess of the connector.

A system for mounting a medical implant, preferably a self-expandable or self-expanding prosthesis, to a delivery catheter configured for delivering the self-expandable or self-expanding prosthesis to an implantation site, wherein the system includes:
the medical implant, preferably the self-expandable or self-expanding prosthesis, wherein the medical implant, preferably the self-expandable or self-expanding prosthesis, includes a self-expandable or self-expanding scaffold;
a delivery catheter configured to deliver the medical implant, preferably the self-expandable or self-expanding prosthesis, to an implantation site, wherein the delivery catheter includes a capsule for covering the medical implant, preferably the self-expandable or self-expanding prosthesis, and optionally a connector, preferably according to one of the preceding examples, for connecting the delivery catheter to the medical implant, preferably the self-expandable or self-expanding prosthesis;
a loading cone for arranging a proximal end portion of the scaffold in a crimped state by decreasing an outer diameter of the proximal end portion of the scaffold, wherein the loading cone includes a body including a distal opening for receiving the scaffold and an opposing proximal opening, wherein the distal opening includes a diameter that is larger than a diameter of the proximal opening, and wherein the body of the loading cone further includes an inner surface extending from the distal opening to the proximal opening, wherein the inner surface tapers towards the proximal opening;
a loading tube configured to be inserted into the proximal opening of the loading cone and to enclose the capsule of the delivery catheter;
   wherein the loading cone and the loading tube are pre-mounted on the delivery catheter, and wherein the system includes a packaging enclosing the delivery catheter as well as the loading cone and the loading tube pre-mounted on the delivery catheter.

The system can include a carrier, wherein the medical implant, preferably the self-expandable or self-expanding prosthesis, is pre-mounted to the carrier.

The system can include a jar for storing the medical implant, preferably the self-expandable or self-expanding prosthesis, being pre-mounted to the carrier.

The medical implant, can be a self-expandable or self-expanding prosthesis, which includes at least one connecting element.

The delivery catheter can include more than one connector and the medical implant, preferably the self-expandable or self-expanding prosthesis, includes more than one connecting element.

The proximal end portion of the scaffold can include one connecting element, which is configured to engage with the corresponding recess of the connector to connect the medical implant, preferably the self-expandable or self-expanding prosthesis, to the delivery catheter.

The proximal end portion of the scaffold can include several connecting elements, and wherein the delivery catheter includes a connector, wherein each connecting element is configured to engage with a corresponding recess of the connector to connect the medical implant, preferably the self-expandable or self-expanding prosthesis, to the delivery catheter.

The loading cone can be configured for receiving the medical implant, preferably the self-expandable or self-expanding prosthesis, pre-mounted to the carrier through the distal opening of the loading cone with the proximal end portion of the scaffold ahead so that the proximal end portion of the scaffold transitions to the crimped state upon sliding along the inner surface of the loading cone and protrudes out of the proximal opening of the body of the loading cone.

The carrier can include a plurality of posts protruding from a base plate of the carrier, wherein the posts are configured to secure the medical implant, preferably the self-expandable or self-expanding prosthesis, to the carrier, and wherein the respective post is configured to be inserted into an associated slot formed in the body of the loading cone for guiding the carrier when the medical implant, preferably the self-expandable or self-expanding prosthesis, is being received by the loading cone.

The medical implant, preferably the self-expandable or self-expanding prosthesis, can be configured to be released from the posts upon sliding along the inner surface of the loading cone to protrude out of the proximal opening of the loading cone with the proximal end portion of the scaffold.

The system can include a splay tube device that is configured to spread the connecting elements apart from one another when the proximal end portion of the scaffold is in the crimped state and protrudes out of the proximal opening of the body of the loading cone so that each connecting element is able to engage with the corresponding recess of the connector.

The splay tube device can include a spring retainer configured to be connected to the carrier, wherein the splay tube device further includes a splay tube that is movably arranged with respect to the spring retainer, wherein the splay tube can be moved from a retracted position into an advanced position when the spring retainer is connected to the carrier, wherein in the advanced position the splay tube extends through the loading cone and protrudes out of the proximal opening of body of the loading cone to spread the connecting elements apart from one another, wherein the spring retainer holds a spring element that is configured to be pretensioned against the splay tube when the latter is moved into the advanced position so that the spring element generates a restoring force that tries to move the splay tube back to the retracted position.

The splay tube device can form a handle when connected to the carrier for removing the carrier and the medical implant, preferably the self-expandable or self-expanding prosthesis, pre-mounted thereon from the jar.

The loading cone can include a loading tube guide connected via at least one spacer bridge to the body of the loading cone, wherein the loading tube guide forms a guiding opening for receiving the loading tube, which guiding opening is aligned with the proximal opening of the body of the loading cone.

The proximal opening of the body of the loading cone can include a ledge that forms a stop for the loading tube.

The loading tube can include a head and an end portion connected to the head, wherein the end portion includes a ledge on an inner surface of the loading tube, which ledge is configured to prevent the capsule from traveling into the loading cone, and wherein optionally the loading tube includes slots formed into an end of the loading tube, which slots allow the end to spread for letting a nose cone of the delivery catheter pass through the loading tube.

The inner surface can taper continuously from the distal opening to the proximal opening.

The inner surface can have a cone shape or conical shape.

A method for connecting a medical implant, preferably a self-expandable or self-expanding prosthesis, to a delivery catheter using a system according to one of the above examples, wherein the method includes the steps of:
inserting the medical implant, preferably the self-expandable or self-expanding prosthesis, pre-mounted to the carrier with the proximal end portion of the scaffold of the medical implant, preferably the self-expandable or self-expanding prosthesis, ahead into the loading cone through the distal opening of the loading cone so that the proximal end portion of the scaffold transitions to the crimped state upon sliding along the inner surface of the loading cone and protrudes out of the proximal opening of the body of the loading cone,
moving the splay tube into its advanced position to spread the connecting elements of the proximal end portion of the scaffold apart and aligning the connecting elements with the recesses of the connector, and moving the splay tube into the retracted position to let the connecting elements engage with the recesses of the connector,
moving the loading tube guided by the loading cone into the proximal opening of the body of the loading cone to cover the connecting elements being engaged with the recesses of the connector to prevent the connecting elements from moving out of the recesses of the connector, and
moving the capsule into the loading tube to cover the medical implant, preferably the self-expandable or self-expanding prosthesis, and pulling the loading tube out of the proximal opening of the body of the loading cone and extracting both loading tube and loading cone from the delivery catheter.

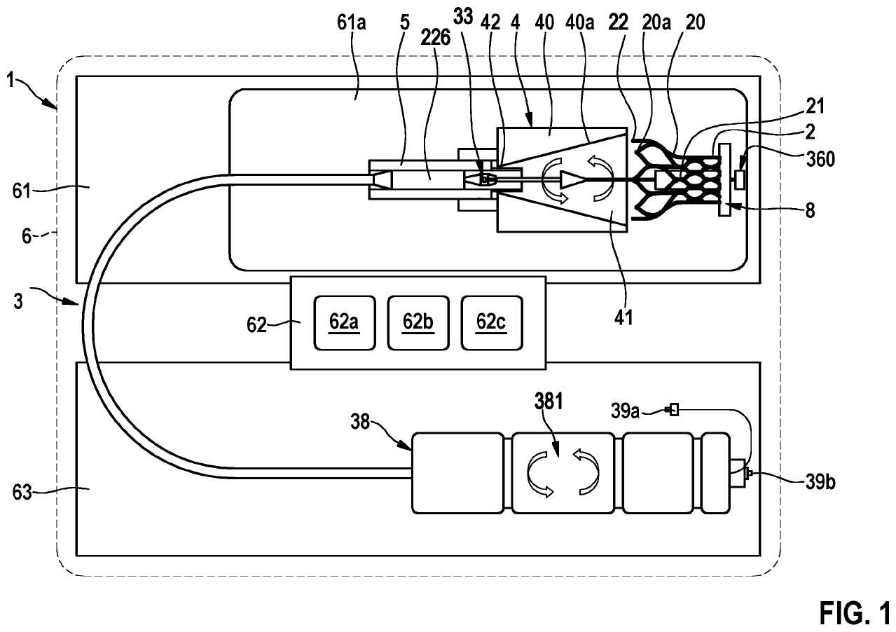

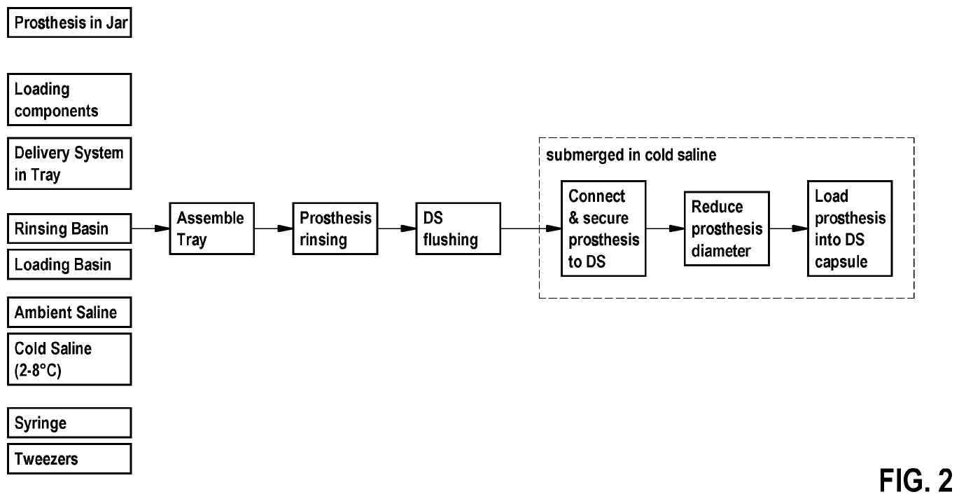

FIG. 1 shows a schematic illustration of an embodiment of a system 1 according to the present invention that is configured for mounting a medical implant 2 to a delivery catheter 3. The medical implant 2 preferably is a self-expandable or self-expanding scaffold 20, e.g. an aortic heart valve prosthesis, wherein the prosthesis and the delivery catheter 3 are preferably configured for a transcatheter aortic valve implantation (TAVI). The delivery catheter 3 is configured to deliver the medical implant 2 to an implantation site. The delivery catheter 3 includes a capsule 226 for covering the medical implant 2, a loading cone 4 and a loading tube 5.

The loading cone 4 is configured to arrange a proximal end portion 20a of the scaffold 20 in a crimped state by decreasing an outer diameter of the proximal end portion 20a of the scaffold 20. The loading cone 4 includes a body 40 including a distal opening 41 for receiving the scaffold 20 and an opposing proximal opening 42, wherein the distal opening 41 includes a diameter that is larger than a diameter of the proximal opening 42. The body 40 of the loading cone 4 further includes an inner surface 40a extending from the distal opening 41 to the proximal opening 42, wherein the inner surface 40 tapers towards the proximal opening 42. The loading tube 5 is configured to be inserted into the proximal opening 42 of the loading cone 4 and to enclose the capsule 226 of the delivery catheter 3. The loading cone 4 and the loading tube 5 are pre-mounted on the delivery catheter 3.

The system 1 further includes a packaging 6 enclosing the delivery catheter 3 as well as the loading cone 4 and the loading tube 5 pre-mounted on the delivery catheter 3. The packaging 6 includes a first packaging part 61, including a loading basin 61a, and a second packaging part 63, including a handle compartment and a splay tube device compartment. The first packaging part 61 and the second packaging part 63 are connectable to each other by a detachable connecting rinsing tray 62.

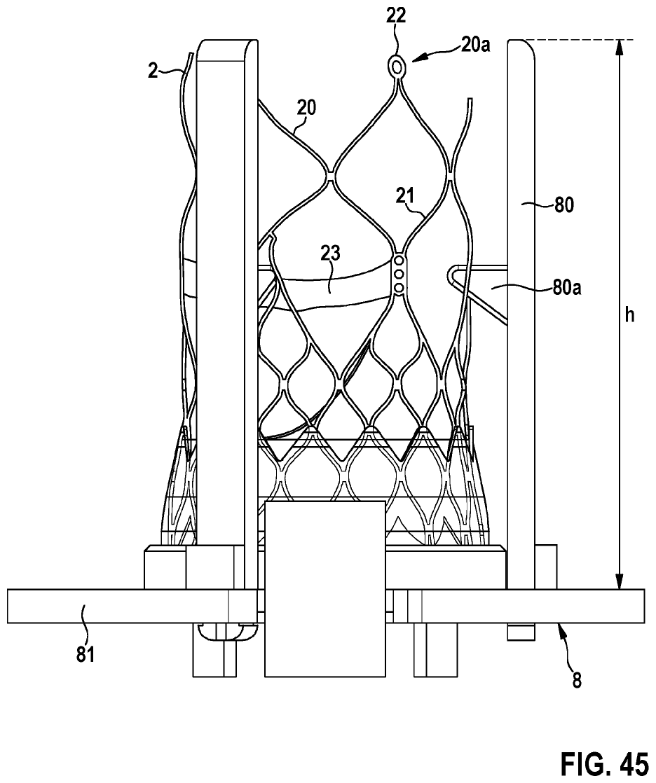

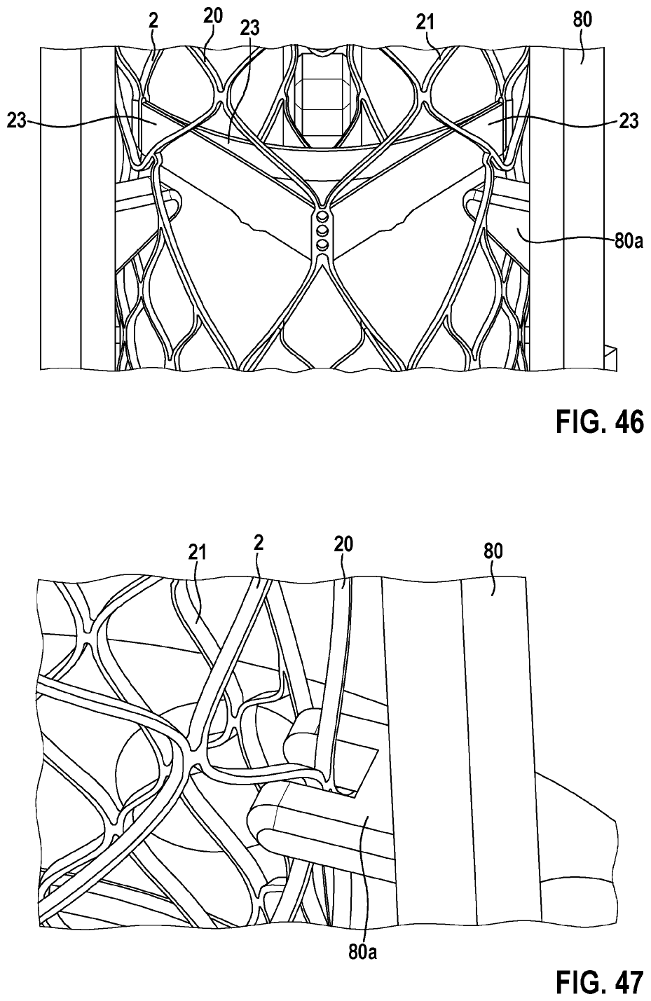

The implant 2 preferably includes a self-expandable scaffold 20 (also denoted as stent) formed out of interconnected struts 21 that are connected to connecting elements 22 at a proximal end portion 20a of the scaffold. The scaffold can carry valve leaflets that can be formed out of a biological tissue (cf. e.g. FIGS. 45 and 46). The scaffold 20 can be formed out of a suitable metal alloy such as a nickel titanium alloy (e.g. Nitinol).

Furthermore, the delivery catheter 3 is configured to deliver the medical implant 2 to an implantation site (preferably via TAVI), wherein the delivery catheter 3 includes a capsule 226 for covering the medical implant 2. The catheter 3 can include a flush port 39a for flushing the catheter 3, and a guidewire lumen flush port 39b for flushing a guidewire lumen of the catheter 3. A stiletto 360 can protects the guidewire lumen from kinking.

The system 1 further includes a loading cone 4 for arranging the proximal end portion 20a of the scaffold 20 in a crimped state by decreasing an outer diameter of the proximal end portion 20a of the scaffold 20, wherein the loading cone 4 includes a body 40 including a distal opening 41 for receiving the scaffold 20 and an opposing proximal opening 42, wherein the distal opening 41 includes a diameter that is larger than a diameter of the proximal opening 42, and wherein the body 40 of the loading cone 4 further includes an inner surface 40a extending from the distal opening 41 to the proximal opening 42, wherein the inner surface 40a tapers towards the proximal opening 42.

Furthermore, the system 1 includes a loading tube 5 configured to be inserted into the proximal opening 42 of the loading cone 4 and to enclose the capsule 226 of the delivery catheter 3, wherein the loading cone 4 and the loading tube 5 are pre-mounted on the delivery catheter 3, and wherein the system 1 includes a packaging 6 enclosing the delivery catheter 3 as well as the loading cone 4 and the loading tube 5 pre-mounted on the delivery catheter 3.

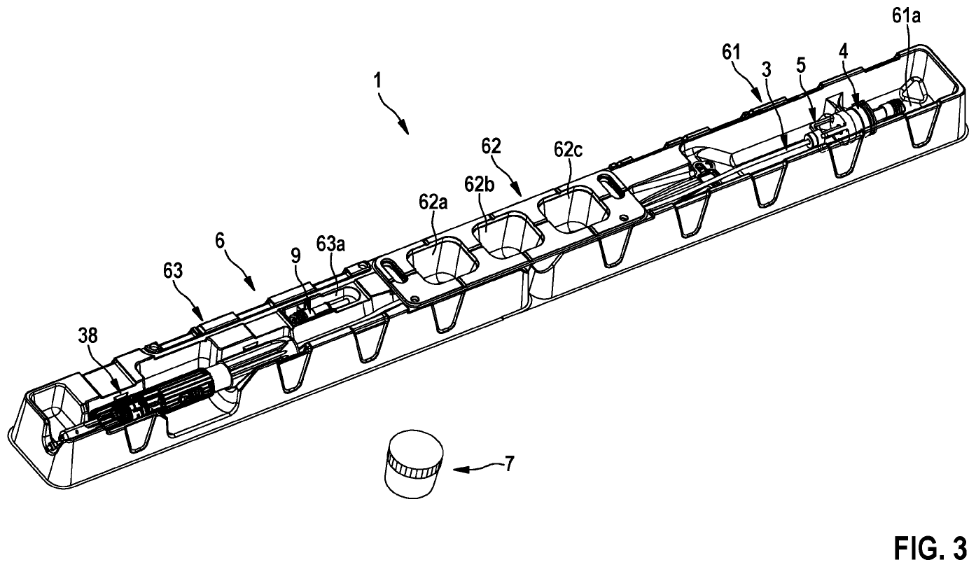

Furthermore, for handling of the medical implant 2, the system preferably further includes an implant storage jar 7 for storing the implant 2 that is preferably pre-mounted to a carrier 8. For removing the carrier 8 and implant 2 from the implant storage jar 7 and for aligning the connecting elements 22 of the scaffold 20, the system 1 can further include a splay tube device 9 (cf. e.g. FIGS. 3 to 5).

Particularly, as indicated in FIGS. 1 and 3, for the loading procedure, the (loading) systems 1 components are initially preferably stored in the following places:
The loading cone 4 is preferably pre-mounted in the first packaging part 61 of the packaging 6,
The loading tube 5 is preferably placed on the delivery catheter's 3 capsule 226 (capsule 226 in open position) and both are preferably placed in the loading cone 4.
The splay tube device 9 is preferably placed in a splay tube device compartment 63a of a second packaging part 63 of the packaging 6.
The prosthesis carrier 8 pre-mounted to the implant 2 is stored in the implant storage jar 7.

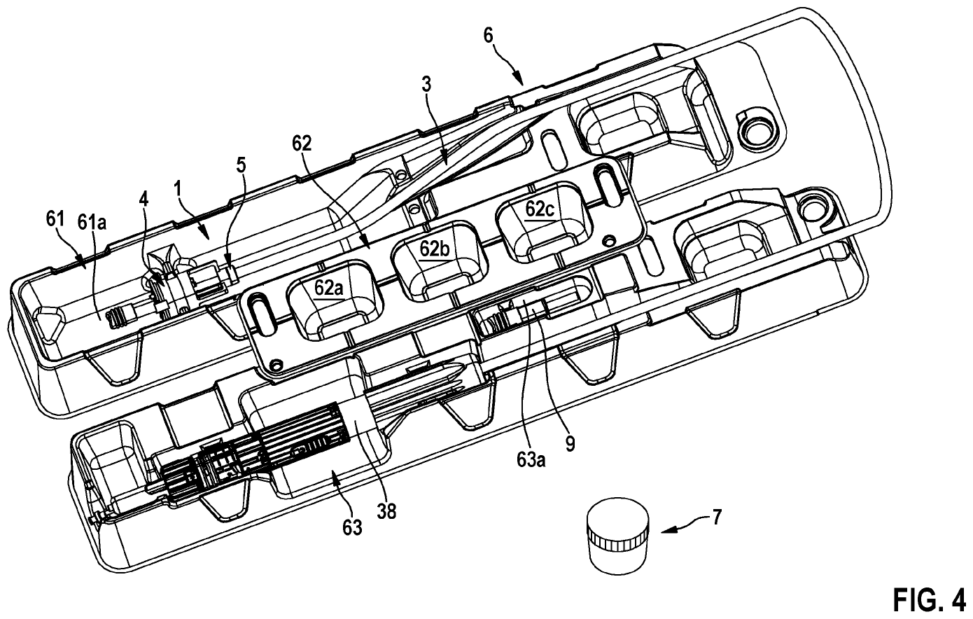

The following sections describe a particular example of the mounting procedure of the medical implant 2 step by step. In a first step, the tray is arranged starting from the initial elongated configuration shown in FIG. 3 to the mounting position shown in FIGS. 1 and 4, wherein a first packaging part 61 of the packaging 6 for receiving the implant 2 or the distal part of the catheter and handle tray 63a for holding a handle 38 of the delivery catheter 3 are arranged on either side of the detachable connecting rinsing tray 62, which contains three rinsing basins 62a, 62b, 62c for rinsing the implant 2.

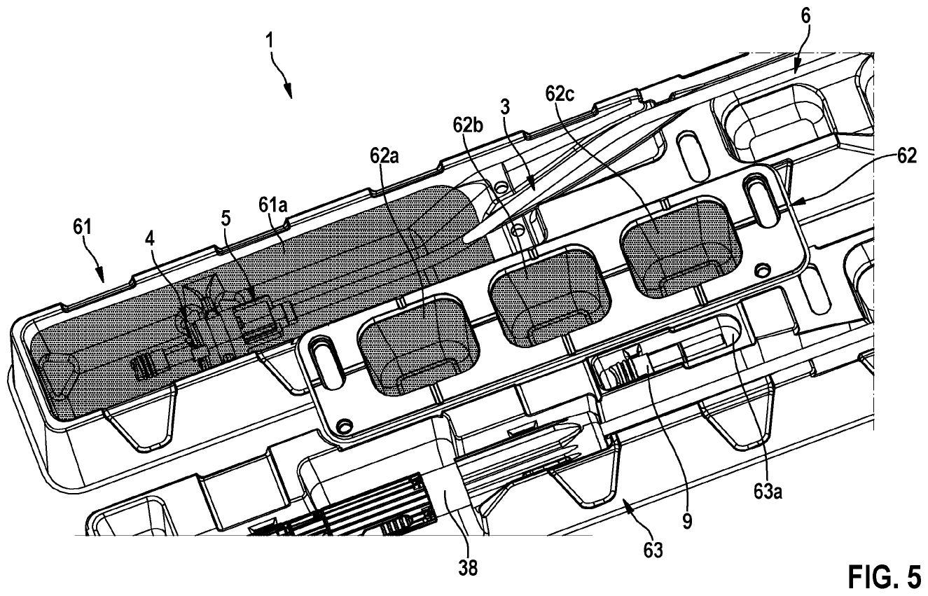

FIG. 4 shows a perspective view of the packaging shown in FIG. 3 in an operating state, wherein the detachable connecting rinsing tray 62 including three rinsing basis 62a, 62b, 62c for storing a saline solution is arranged between the first packaging part 61 for holding a distal portion of the delivery catheter to which a self-expandable medical implant, e.g. a self-expandable aortic heart valve prosthesis, shall be mounted and the second packaging part 63 for holding a handle 38 of the delivery catheter 3. The handle may have a deployment wheel 381. After having placed the packaging 6 in the configuration shown in FIG. 4, particularly, the three rinsing basins 62a, 62b, 62c of the detachable connecting rinsing tray 62 are preferably filled with saline at room temperature (cf. FIG. 5). Furthermore, the loading basin 61a is preferably filled with (physiological) saline having a temperature of between 0° C. and 8° C. (cf. FIG. 5).

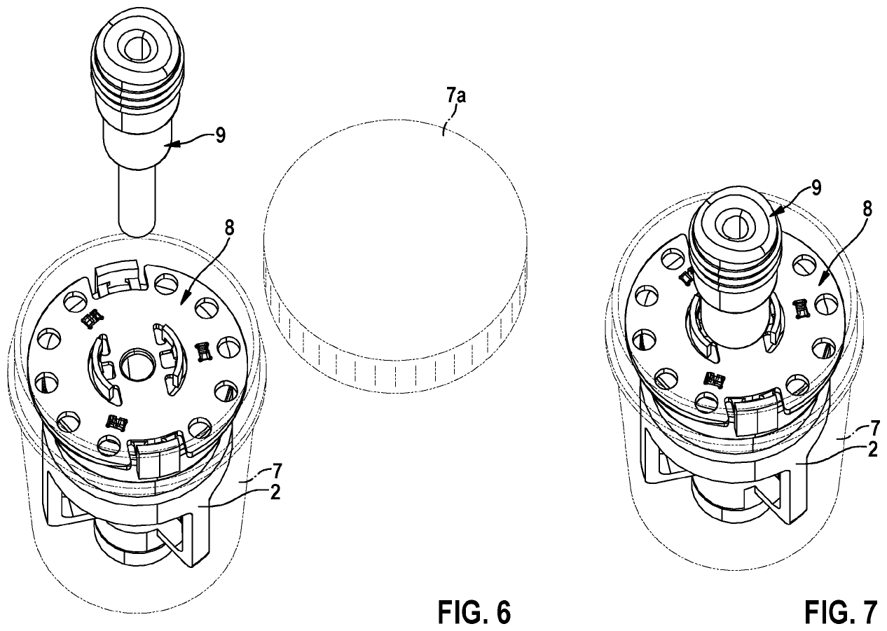

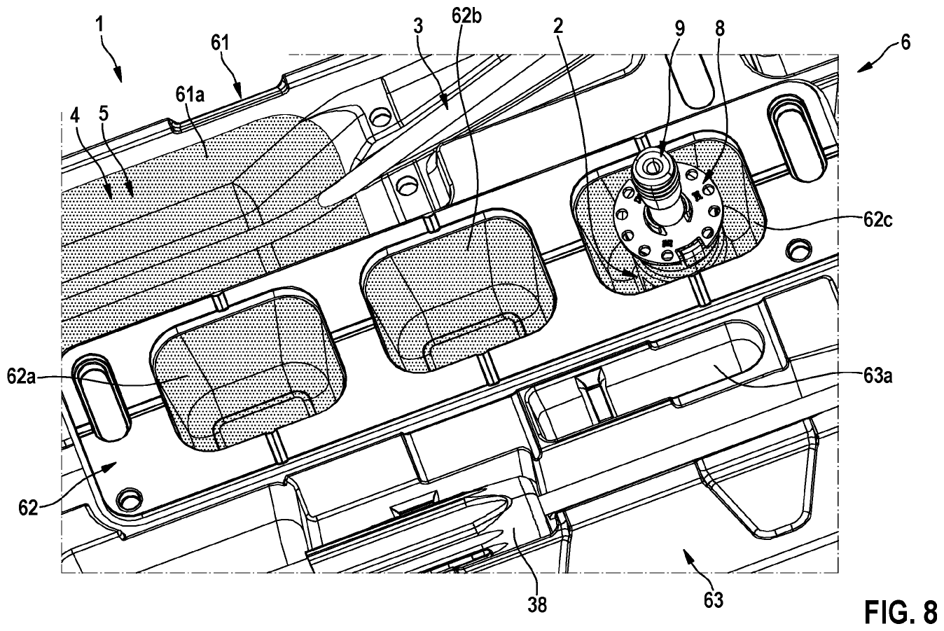

FIG. 6 shows a perspective view of an implant storage jar 7 including an implant 2 and an implant storage jar lid 7a and a splay tube 9 in a not assembled state. The implant 2 is pre-mounted on a carrier 8. The splay tube device 9 (having been arranged in the second packaging part 63) is used to extract the implant 2, e.g. the prosthesis, from the implant storage jar 7, and the rinsing process is started, wherein the implant 2, e.g. the prosthesis, is preferably rinsed in each rinsing basin 62a, 62b, 62c for e.g. 1 minute before moving to the next one.

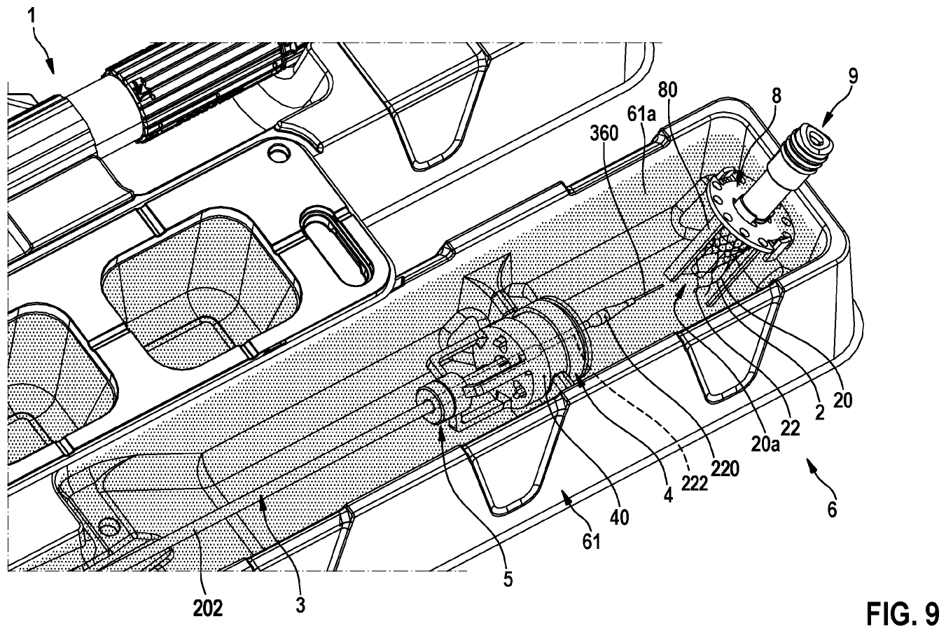

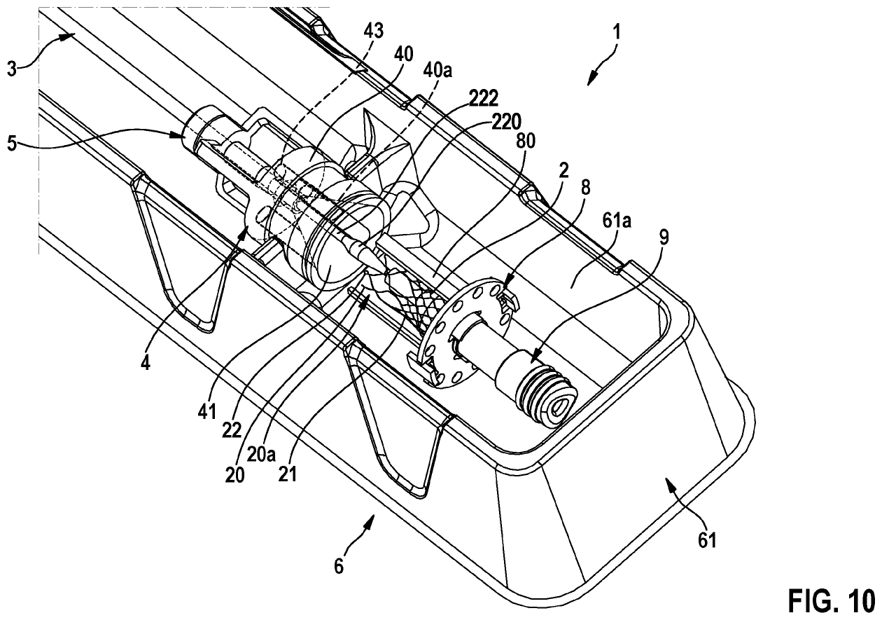

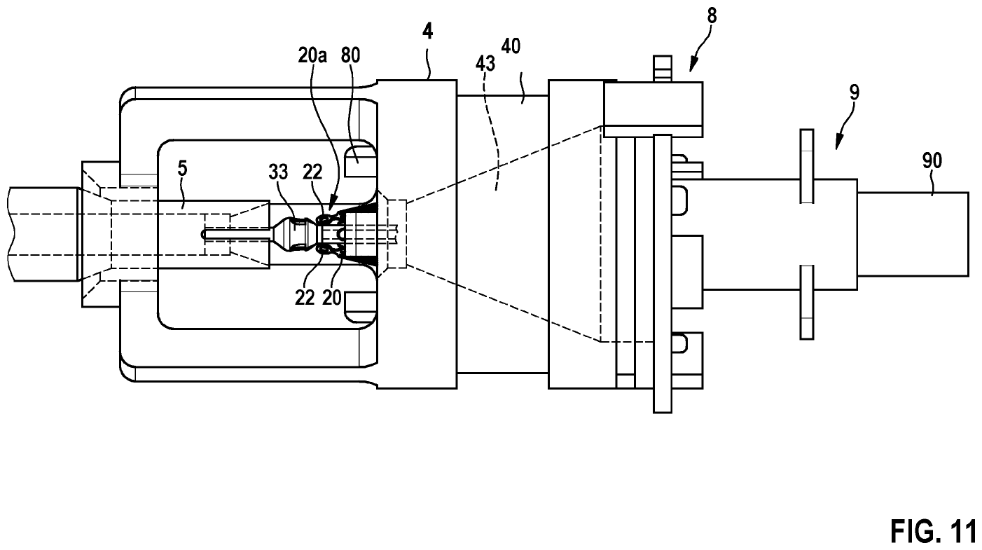

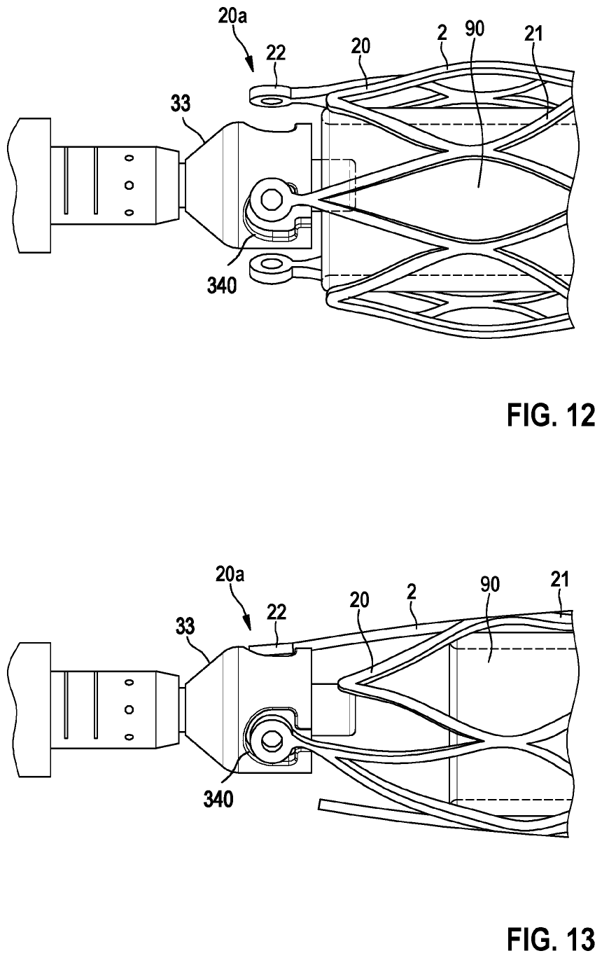

FIG. 7 shows a perspective view of the implant storage jar 7 of FIG. 6 including an implant 2 connected to a carrier 8 and a splay tube 9 being connected to the carrier. When the splay tube device 9 is connected to the carrier 8 the medical implant 2 can be inserted into a loading cone of the system 1. Thereafter, the capsule 226 in the loading basin 61a and the stabilizer lumen of the delivery catheter 3 is flushed with saline. The carrier 8 with the implant pre-mounted thereon is then inserted into the loading cone 4 (cf. FIGS. 9 and 10). The carrier 8 is submerged in the saline and the nose cone 220 and the stiletto 360 (which protects the guidewire lumen 222 from kinking) is guided into the splay tube device 9. The carrier 8 is moved towards the loading cone 4 and can be rotated to align the posts 80 of the carrier 8 with the three slots 43 formed in the body 40 of the loading cone 4. The carrier 8 is then slid forward until it is secured by a snap fit (alternative connection methods will be described further below) between carrier 8 and loading cone 4, thus crimping down the proximal end portion 20a (outflow of the implant 2) of the scaffold 20 to a smaller diameter. The loading tube 5 is pulled back as shown in FIG. 11 so that the connector 33 is accessible. The splay tube 90 is now pushed forward to spread the connecting elements 22 (e.g. having eyelets) of the scaffold apart in the radial direction. As indicated in FIGS. 12 and 13, the connecting elements 22 are aligned with the recesses 340 in the connector 33 by e.g. rotating the loading cone 4 and the carrier 8. The splay tube 90 is then released/retracted to connect the connecting elements 22 to the connector 33. To facilitate the alignment of the connecting elements, a mirror can be attached to the bottom of the loading basin 61a. Alternatively, the delivery catheter 3 can include a sloped connector 33 as will be described further below.

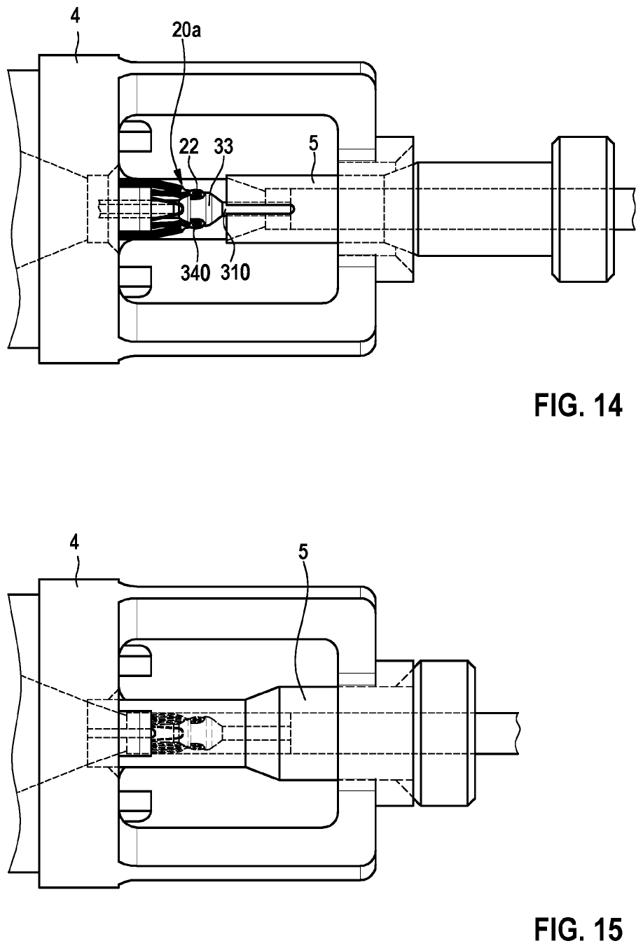

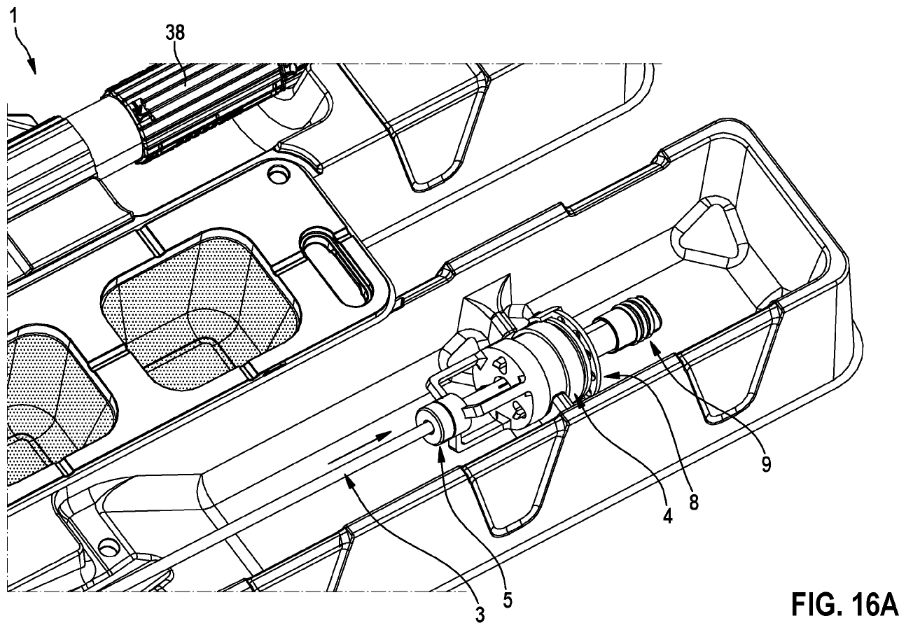

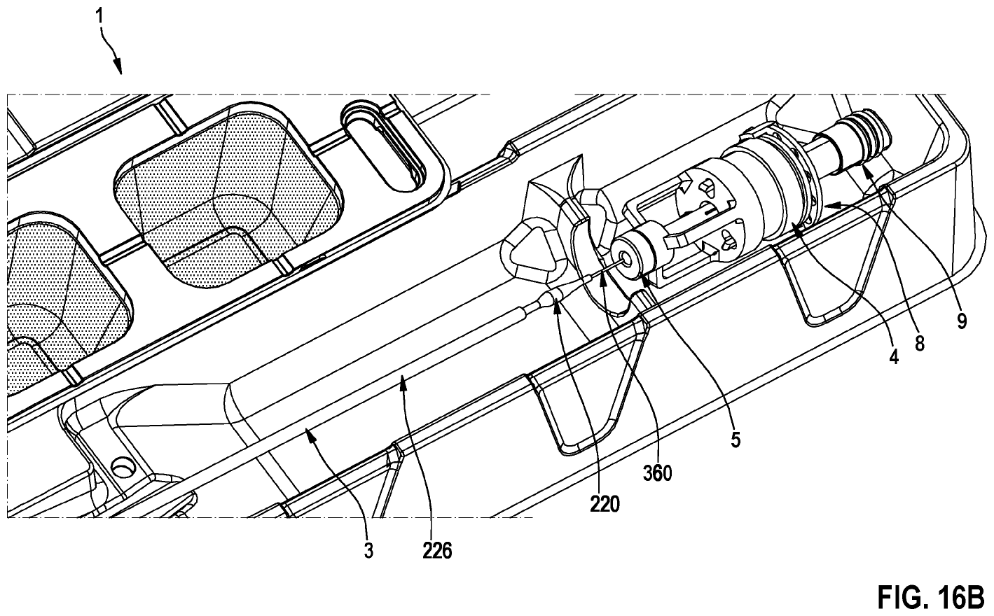

As shown in FIGS. 14 and 15, the loading tube 5 is now pushed forward over the connector 33 and connecting elements 22 engaged therewith until it hits a hard stop to secure the prosthesis connecting elements 22 in the connector 33. The connector 33 has recesses 340 for engaging with the connecting elements 22, which are not located in the distal part 310 of the connector. Afterwards, the capsule 226 is closed by rotating a delivery catheter actuator on the handle 38. The capsule 226 will reach a hard stop when it is fully closed (cf. FIG. 16A). Thereafter, as shown in FIG. 16B, the loading tube 5 is pulled out of the loading cone 4 and both the loading tube 5 and the loading cone 4 are removed from the delivery catheter 3. Pulling out the loading tube 5 allows the nose cone 220 to pass through the loading tube. Finally, the stiletto 360 is removed and the guidewire lumen 222 is flushed.

Particularly, according to an alternative embodiment, the connector 33 could be attached to the connecting elements 22 before submerging it in (physiological) saline having a temperature of between 0° C. and 8° C. to prevent staying deformation of the connecting elements 22. Furthermore, instead of pulling the medical implant (e.g. prosthesis) 2 directly into the capsule 226, an intermediate step could be introduced, where the medical implant (e.g. prosthesis) 2 is pulled back into the loading tube 5 and is encapsulated from there.

The medical implant (e.g. prosthesis) 2 would have to be secured by slightly moving forward the capsule 226 and securing the medical implant (e.g. prosthesis) 2, then pulling it into the loading tube 5 by pulling on the outer sheath 202 of the delivery catheter 3. Furthermore, instead of extracting the loading tube 5 and the loading cone 4 from the delivery catheter 3 one could also pull the delivery catheter 3 out of the (loading) system 1. Furthermore, the duration of the rinsing steps could potentially be reduced to save time during the loading process. Further, the mounting of the implant 2 could also be performed in saline at room temperature.

In the following, the components of the system and embodiments relating thereto will be described in more detail.

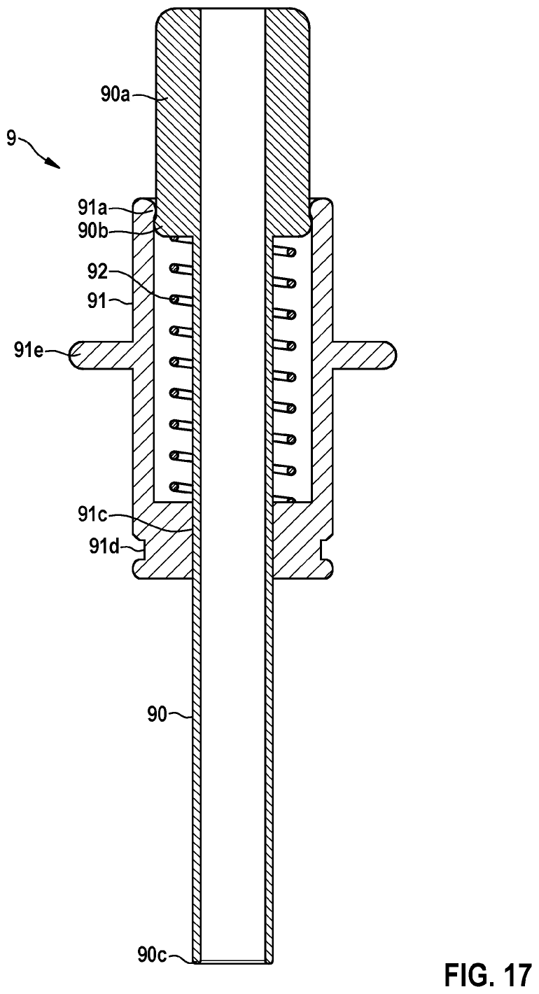

FIG. 17 shows an embodiment of a splay tube device 9 that can be used with a system 1 according to the present invention. The splay tube device 9 includes three parts, a spring retainer 91, a spring element 92 and a splay tube 90. The spring element 92 pretensions the splay tube 90 when the splay tube 90 is moved to an advanced position as can be inferred from FIG. 17. This can be achieved by pushing down on the head 90a of the splay tube 90 tube. Particularly, the splay tube 90 has two positions, a retracted or resting position and the advanced position, where the splay tube 90 is extended to spread the connecting elements (e.g. eyelets) 22 of the medical implant (e.g. prosthesis) 2 within the loading cone 4. The spring retainer's 91 main function is to work as a counterpart to the splay tube 90. By retaining the spring element 92 within the retainer 91 it allows the splay tube 90 to extend and thereby to tension. Furthermore, the spring retainer 91 is configured to be connected to the carrier 8 and forms a handle to aid during the extraction and flushing process.

Furthermore, the spring retainer 91 includes a lip 91a that prevents the splay tube 90 from escaping. The splay tube 90 includes a corresponding lip 90b which overlaps with the lip 91a of the spring retainer 91. These parts are connected by pressure, extending the radius of the spring retainer lip 91a. The force for disassembling the splay tube 90 is high enough so it does not detach during transport and use. The spring retainer 91 further includes a tube passage 91c that allows the splay tube 90 to reach trough the spring retainer 91 and also acts as a guide to keep the spring retainer 91 and the splay tube 90 axially aligned. The larger the height of this passage 91c, the better the alignment of splay tube 90 and retainer 91. Furthermore, the retainer 91 can include a circumferential groove 91d to connect the splay tube device 9 to the carrier 8, e.g. via latching noses of the carrier 8 that engage with the groove to establish a snap-fit connection. This connection and possible alternatives will be described in more detail below.

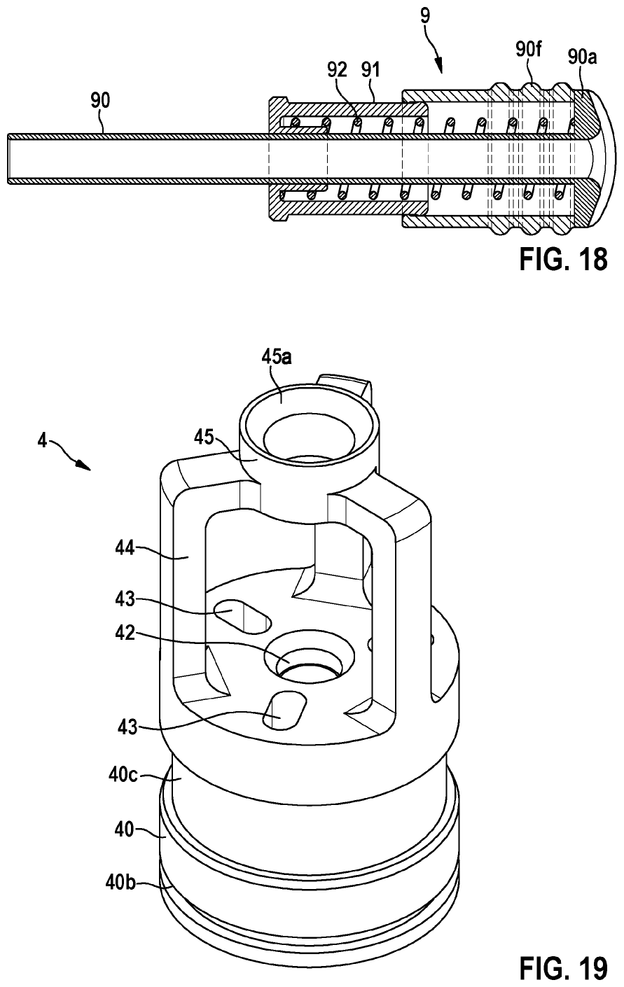

Furthermore, the retainer 91 can include a finger flange 91e, that allows the spring retainer 91 to be secured while pushing forward the splay tube 90. Particularly, this mechanism functions similar to one seen in a syringe. As indicated in FIG. 17, the splay tube 90 is hollow to allow the nose cone 220 of the delivery catheter 3 to pass through. The tip 90c of the tube 90 is preferably rounded. This prevents damage to the prosthesis leaflets 23 when introducing the splay tube 90 into the prosthesis carrier 8. This tip 90c of the tube 90 is the part that spreads the connecting elements 22 (e.g. eyelets) of the medical implant (e.g. prosthesis) 2 when the splay tube 90 is extended to position it in its advanced position. As an alternative, the splay tube 90 can also have a larger diameter than the spring retainer 91. In this way the spring retainer 91 would fit inside the splay tube 90. Further, instead of the finger flanges 91e as shown in FIG. 17 different profiles 90f (such as e.g. circumferential projections 90f) are feasible to create a better grip (see FIG. 18).

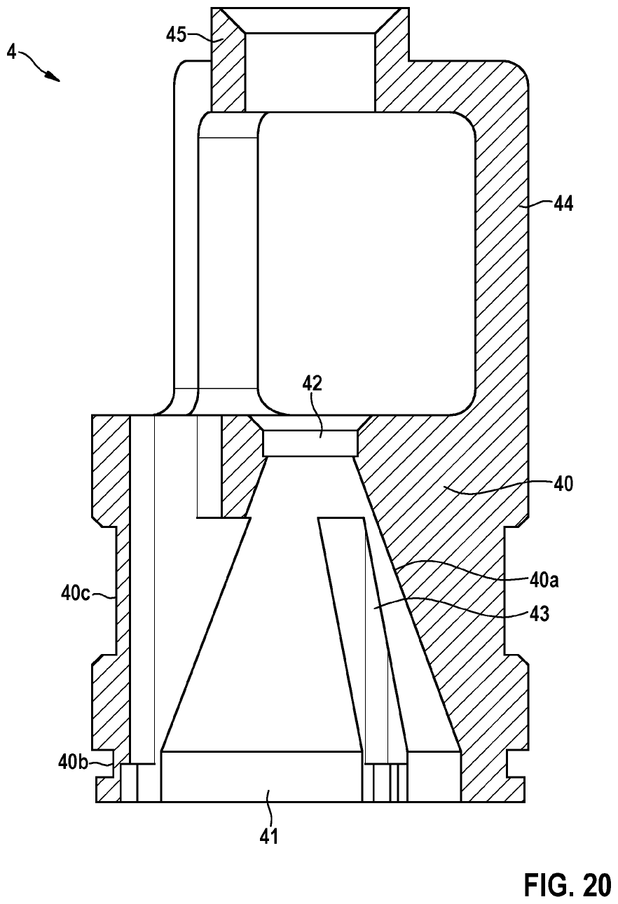

A preferred embodiment of the loading cone 4 is shown in FIGS. 19 and 20. The loading cone 4 includes a body 40 including at a distal loading cone section a distal opening (not visible in FIG. 19) for receiving the scaffold and an opposing proximal opening 42 in the loading cone's middle section, wherein the distal opening includes a diameter that is larger than a diameter of the proximal opening 42. The body 40 of the loading cone 4 further includes an inner surface 40a extending from the distal opening 41 to the proximal opening 42, wherein the inner surface 40a tapers towards the proximal opening 42. The body 40 of the loading cone 4 further includes at least two, preferably three, internal slots 43. A groove 40b is formed on the outer side of the body at the distal loading cone section. The body 40 of the loading cone 4 can include a further circumferential recess 40c for fixing the loading cone 4 in a packaging. The loading cone 4 includes at its proximal section a loading tube guide 45 that is configured to guide the loading tube 5 to the countersunk proximal opening 42 of the body 40 of the loading cone 4. The guide 45 is an annular member 45 that includes a central guiding opening 45a and is connected to the body 40 of the loading cone 4 via at least two, preferably three, spacer bridges 44. The spacer bridges 44 create an empty space between the loading tube guide 45 and the body 40 of the loading cone 4. The loading cone's 4 main purposes are to crimp the outflow 20a of the medical implant (e.g. prosthesis) 2 and help guide it into the loading tube 5 or capsule 226.

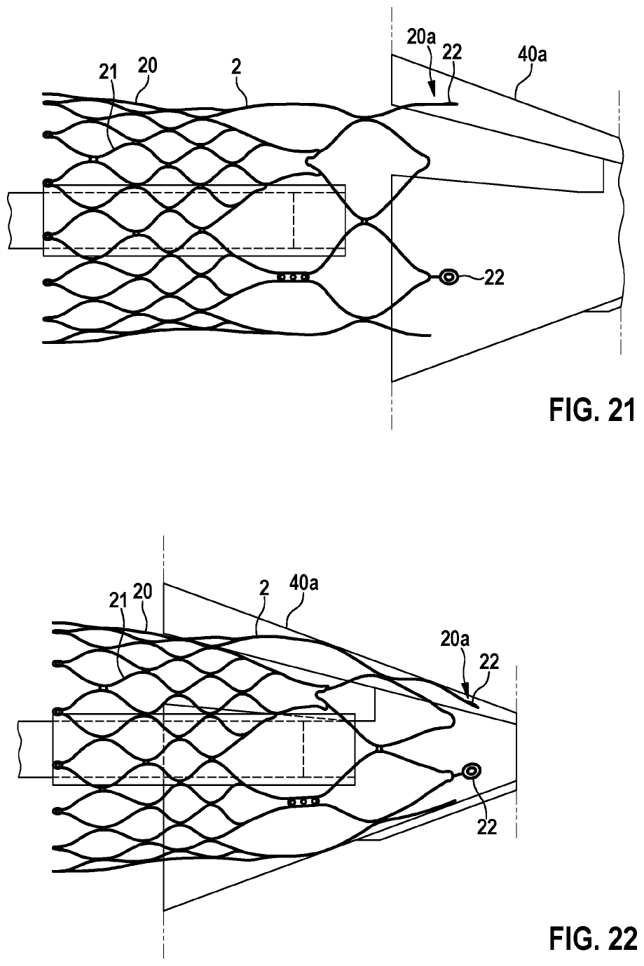

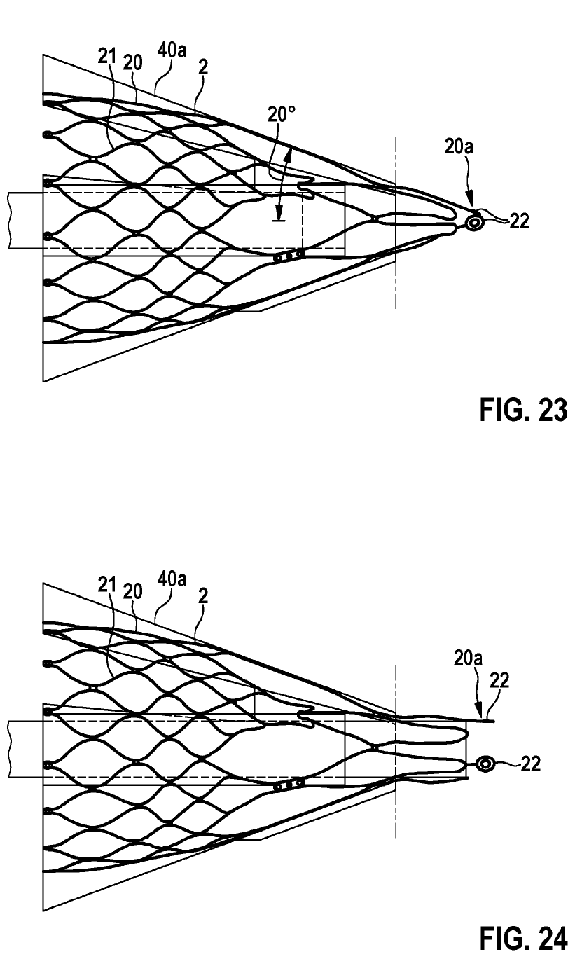

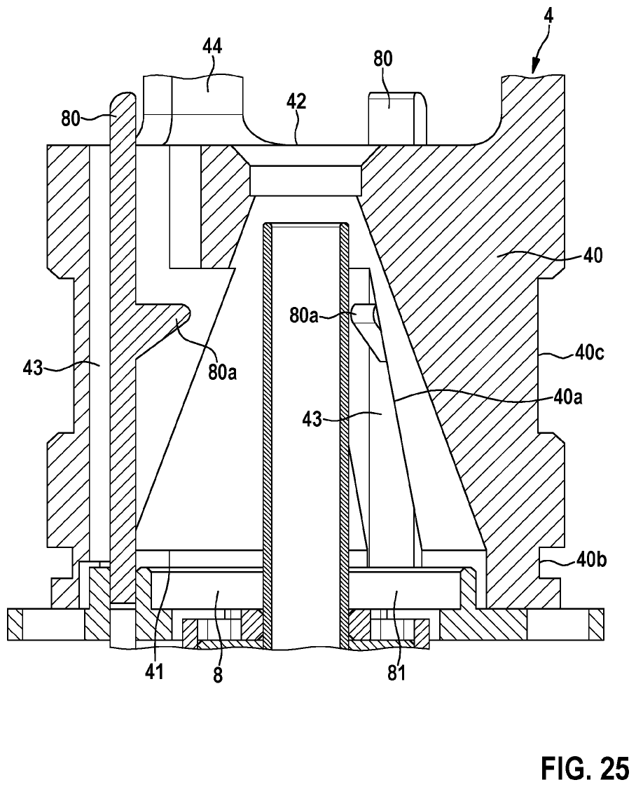

When inserting the prosthesis carrier 8 into loading cone 4 through its distal opening 41, the tapered shape, preferably cone shape, of the inner surface 40a of the body 40 of the loading cone 4, guides the outflow/proximal end portion 20a of the scaffold 20 and the diameter of the outflow/proximal end portion 20a is reduced as shown in FIGS. 21 to 24. The inner surface 40a is arranged at an angle of preferably 20°. (Other angles and shapes e.g. trumpet shapes are feasible) As previously described, temporary spreading of the connecting elements 22, e.g. having eyelets, (with the splay tube 90) allows the connecting elements 22 to be hooked into the connector's 33 recesses 34. Three slots 43 (other amounts of carrier bars are possible) that are formed in the body 40 of the loading cone 4 which are preferably spread in a 120° angle act as socket for the posts 80 of the carrier 8. The prosthesis carrier posts 80 (cf. FIG. 25) help guide the medical implant (e.g. prosthesis) 2 into the loading cone 4. Particularly, they prevent the medical implant (e.g. prosthesis) 2 from rotating in the loading cone 4 and ensure that the loading cone 4 and a base plate 81 of the carrier 8 are aligned axially. During this process the medical implant (e.g. prosthesis) 2 is lifted out of the prosthesis holders 80a by the cone shape of the inner surface 40a of the loading cone 4. The holders 80a are preferably formed as projections protruding from the posts 80 of the carrier 8.

In the following, an embodiment of the carrier 8 and its functionality with regard to holding the implant 2 will be described in more detail.

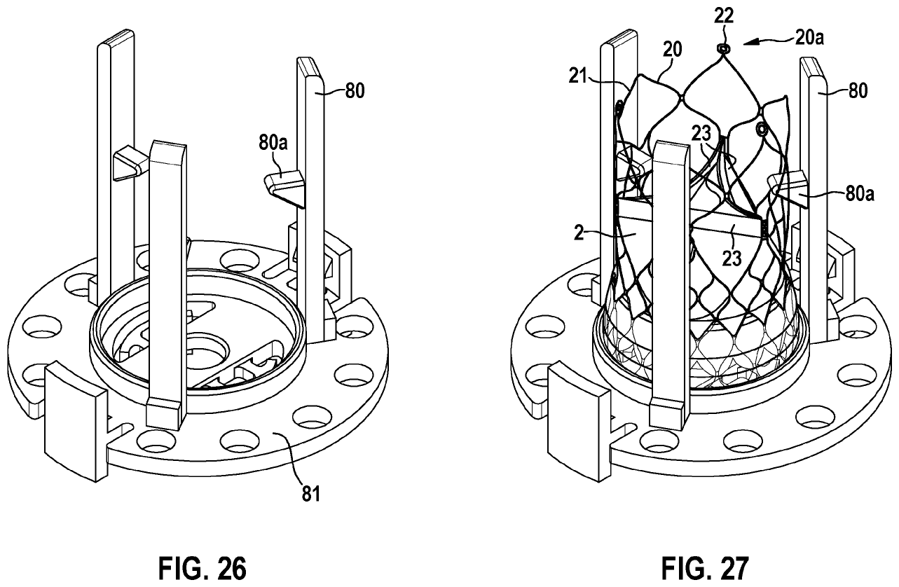

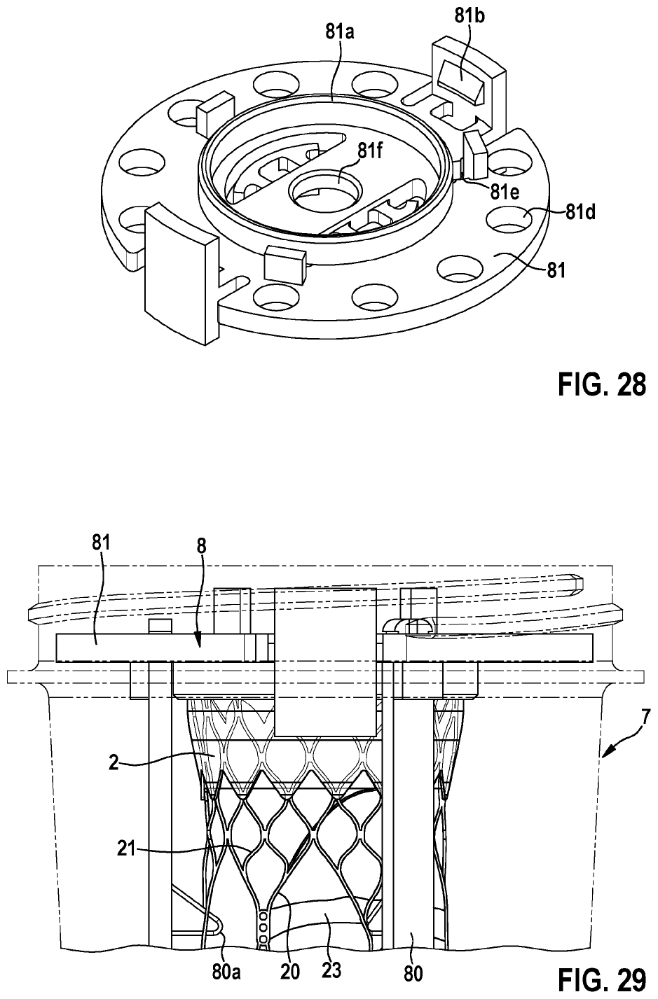

As indicated in FIGS. 26 and 28, the prosthesis carrier 8 can include two basic elements, namely a base plate 81 and particularly three posts 80 for carrying the implant 20. However, it is also possible to form the posts 80 and the base plate 81 in one integral part (e.g. using an injection molding approach). The posts 80 protrude from the base plate 81 and each include a holder 80a (e.g. in form of a nose) that engages with the scaffold 20 as shown in FIG. 27 to hold the implant 2. The noses 80a particularly engage with the scaffold above and/or between the leaflets 23 to protect the latter from damage.

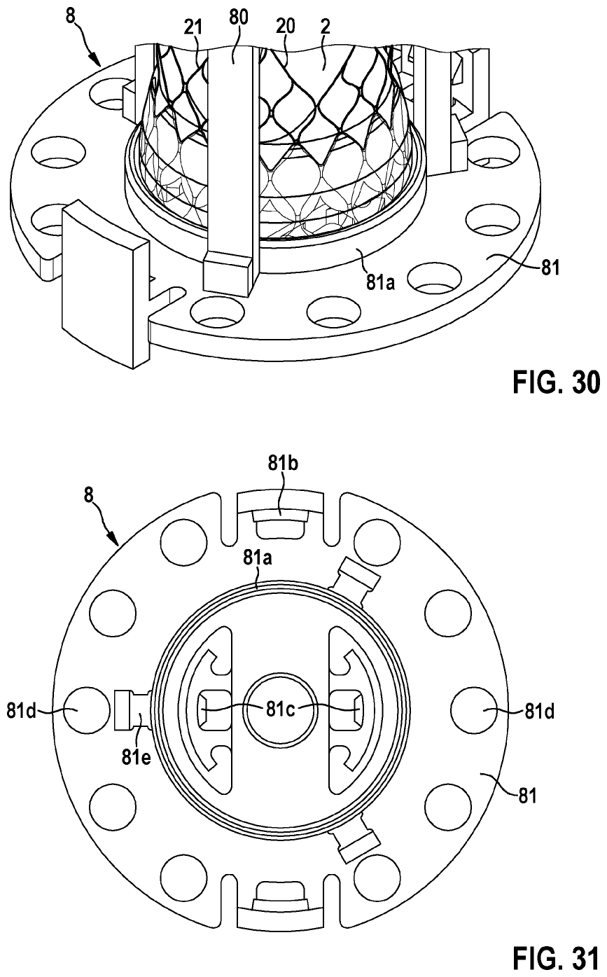

As further shown in FIG. 28, the base plate 81 can be formed as a flat disk 81 including a ring 81a to receive the medical implant (e.g. prosthesis) 22 (cf. FIG. 30), as well as latching noses 81b for attaching the base plate 81 to the body 40 of the loading cone 4 and the splay tube device 9. Furthermore, the base plate 81 includes venting holes 81d so it displaces less water when arranged in the jar 7. Furthermore, the base plate 81 includes fastening holes 81e for receiving the posts 80. The base plate 81 includes a trough-hole 81f in the center of the base plate 81 that forms a passage for the splay tube of the splay tube device.

For storing the implant 2 before mounting it to the delivery catheter 3, the system 1 preferably includes an implant storage jar 7. An embodiment of the implant storage jar 7 is shown in FIG. 29. According thereto, the outer diameter of the base plate 81 of the carrier 8 is sized to sit directly on top of a rim of the prosthesis jar. However, the carrier 8 does not necessarily have to sit on this ring. It could also be attached to the lid or rest on the posts 80 on a bottom of the implant storage jar 7.

According to an alternative embodiment, the carrier 8 could also be combined with the implant storage jar lid 7a of the implant storage jar 7 (either as separate parts or as one part). The splay tube device 9 would have to be left away or could be integrated. The ring 81a in the center of the carrier base plate 81 is used to center the medical implant (e.g. prosthesis) 2. As an alternative, centering ring 81a (or any other centering possibility or shapes) could also be on the inside of the medical implant (e.g. prosthesis) 2 (instead of on the outside) or both on the inside and outside for extra stability.

Preferably, as shown in FIG. 31, the outer part of the prosthesis carrier 8 includes a plurality of holes 81d, e.g. 10 holes. These holes 81d have two functions. They reduce the volume and the solution which is displaced by the prosthesis carrier 8, and further allow the solution to flow through it when placing the medical implant (e.g. prosthesis) 2 into the solution. As an alternative, these circular holes 81d can also be replaced by slots or openings in any other shape or size.

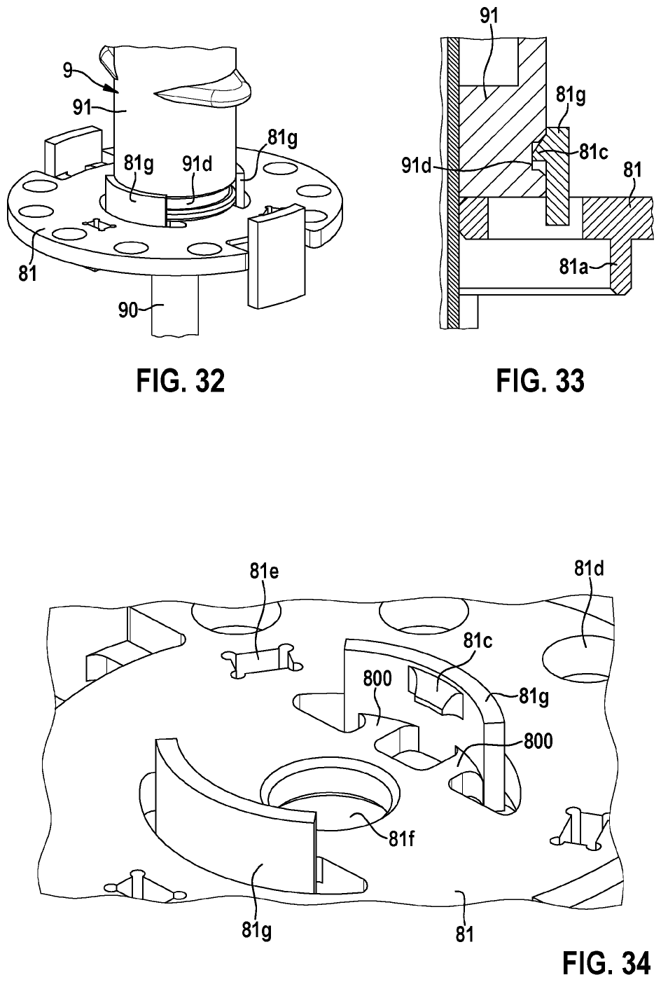

Furthermore, FIGS. 31 to 34 illustrate a snap-fit connection between the splay tube device 9 and the carrier 8 that uses the latching noses 81c of the base plate 81 as introduced above.

As particularly shown in FIG. 34, the base plate 81 includes a trough-hole 81f in the center of the base plate 81 that forms a passage, preferably for the splay tube 90 of the splay tube device 9, which has the function of spreading the connecting elements 22 (e.g. eyelets) during the mounting procedure of the implant 2. (Two) pivotable wings 81g are arranged on either side of the central through-hole 81f, wherein an outer side of the respective wing 81g has a rounded shape in order to guide and center the spring retainer 91 of the splay tube 90. The respective latching nose 81c protrudes from a concave inner side of the respective wing 81g and is configured to interlock with a recess, particularly the groove 91d, of the spring retainer 91 of the splay tube device 9 (cf. FIG. 17) which locks the spring retainer 91 in place with respect to the carrier 8. Furthermore, each wing 81g is arranged in an opening of the base plate 81 and is connected via (two) bridges to the base plate 81 as shown in FIG. 34. The two bridges 800 of each wing/snapper 81g allow the wing 81g and the latching nose 81c connected thereto to pivot out of the way when inserting the spring retainer 90 between the wings 81g to fasten the splay tube device 9 to the plate 81 of the carrier 8. Particularly, the bendable snap-fit wings 81d are designed such that the splay tube device 9 cannot be removed from the base plate 81 once it is inserted. However, according to an alternative embodiment, the splay tube device 9 could also be designed with a removable snap-fit, allowing the user to remove the splay tube device 9 and giving the user more flexibility for the procedure. The latching noses 81c and groove 91d can also be interchanged so that the latching noses are provided on the spring retainer 91 and the groove on the base plate 81. Other options for connecting the splay tube device 9 to the carrier 8 that are also conceivable are: a bayonet connector, a screw thread or a press fit etc.

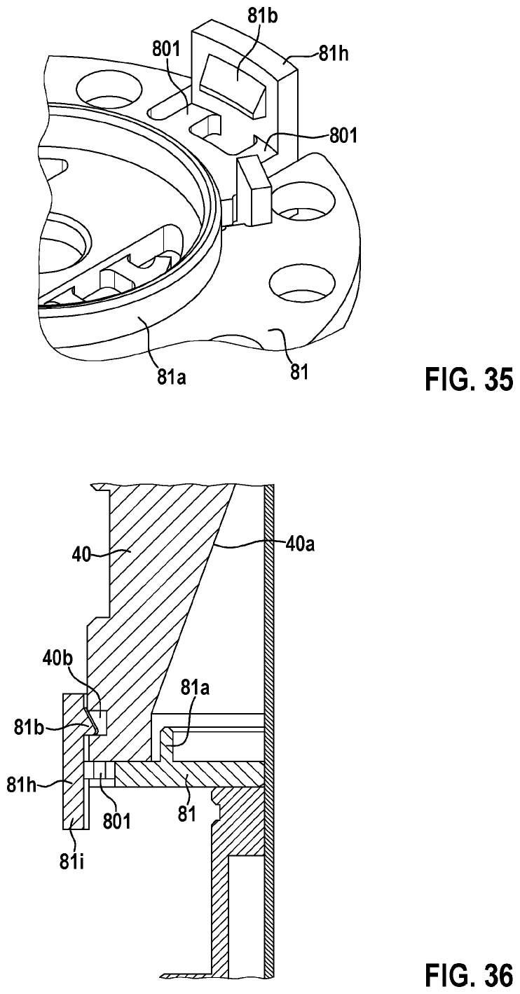

FIGS. 35 and 36 illustrate a snap-fit connection between the base plate 81 of the carrier 8 and the body 40 of the loading cone 4. According thereto, the base plate 81 includes (two opposing) wings 81h for the loading cone 4 that are arranged in recesses of the periphery of the base plate 81 and are each connected to the base plate 81 via (two) bridges 801 so that the respective loading cone wing 81h is pivotable. An outer side of the respective loading cone wing 81h of the base plate 81 has a rounded shape. Each of the latching noses 81b protrudes from an inner side of the respective loading cone wing 81h and is configured to interlock with the groove 40b formed in the body of the loading cone 4 (cf. FIG. 36). The loading cone wings 81h of the base plate 81 and noses 81b thereon are shaped such that the wings 81h bend out of the way when the loading cone is inserted in between the wings 81h and such that the noses 81b engage with the groove 40b to lock the loading cone 4 in place. The two bridges 801 of each loading cone wing 81h allow the respective wing 81h to pivot out of the way when inserting the loading cone 4 in between the wings 81h. The wings 81h are designed such that the base plate 81 can be removed from the loading cone 4 by pressing on the wings 81h to pivot the noses 81b out of the groove 40b.

As an alternative, it is also conceivable to interchange the positions of the latching noses 81b and the wings 81h. Another option for connecting the carrier 8 to the loading cone could be a press fit, magnetic-, screwed-, or wedged-connector. The length of the end portion 81i of the respective wing 81h that serves as a release handle, which end portion 81i is arranged opposite the latching nose 81b, can be further increased to reach the implant jar lid 7a. This would allow for less movement of the implant 2 within the prosthesis jar 7 and would reduce the force needed to release the carrier 8 from the loading cone (cf. FIG. 36).

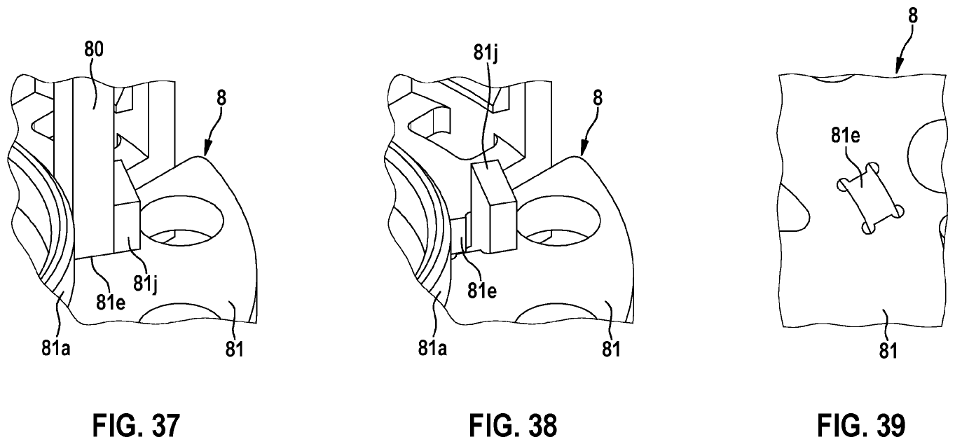

Furthermore, as indicated in FIGS. 37 to 39, for connecting the posts 80 to the base 81 of the carrier 8, the base plate 81 includes three through-holes 81e arranged outside the centering ring 81a. The holes 81e are arranged along the ring 81a 120° apart from one another and are each configured to receive an end portion 80b of the respective post 80. The holes 81e thus form anchoring points for the posts 80. Next to each anchoring hole 81e a support 81j protrudes from the base plate 81. The prosthesis centering ring 81a and the carrier post supports 81j both prevent radial displacement of the respective carrier post 80. The corners of the holes 81e can be drilled out with a 1 mm diameter drill to maintain manufacturability by milling. As an alternative, the supports 81j could extend on all sides of the respective post 80 to further reduce mobility of the posts 80.

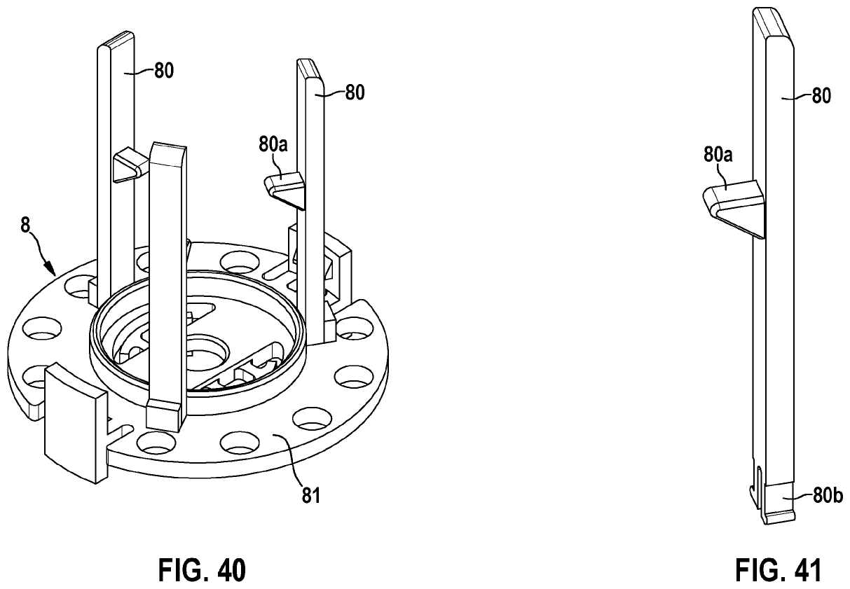

According to FIGS. 40 and 41, the three posts 80 of the carrier 8 are attached to the base plate 8 using a snap-fit connection, respectively. With respect to the periphery of the base plate 81, the posts are arranged 120° apart from one another. Particularly, the posts have the following functions: Securing the medical implant (e.g. prosthesis) 2 to the carrier 8, and guiding the carrier 8 into the loading cone 4. Instead of three posts 80 also a smaller number or a larger number of posts 80 can be used (e.g. 2 or 4 posts 80).

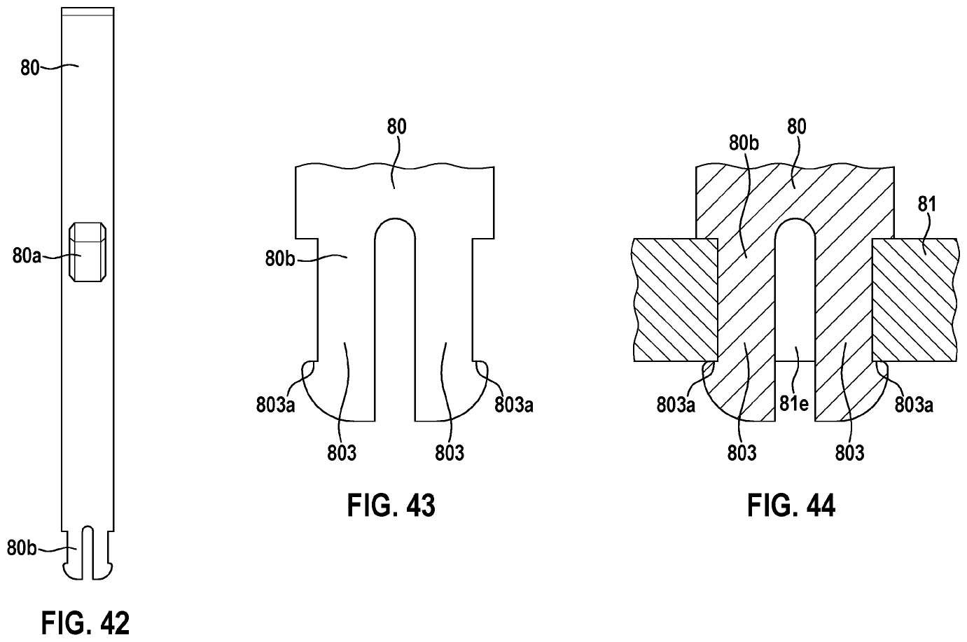

FIGS. 42 to 44 illustrate the snap-fit connection between the respective post 80 and the base plate 81 of the carrier 8. According thereto, the respective post includes an end portion 80b that includes a gap so that the respective end portion 80b includes two bendable legs 803. The gap between the two legs 803 of each post 80 allows the legs 803 to bend inwards. Each leg includes a nose 803a so that it can engage behind the base plate 81 once it is properly arranged in the associated hole 81e of the base plate 81 as shown in FIG. 44. Also, here the positions of the legs 803 and holes 81e can be interchanged according to an alternative embodiment. Furthermore, other methods of connecting the posts 80 to the base plate 81 could involve: a thread, glued posts, screws etc.

Furthermore, as indicated in FIG. 45, the height h of the carrier posts 80 with respect to the base plate 81 is preferably selected such that the posts 80 are higher than the total height of the medical implant (e.g. prosthesis) 2. This prevents the prosthesis outflow 20a from coming in contact with the prosthesis jar 7 (if the fixation of the carrier 8 in the jar is guaranteed, the posts 80 can also be shorter than the medical implant (e.g. prosthesis) 2). As an alternative, the length of the posts could be adjusted to the length of the jar to allow less movement within the jar 7.

As illustrated in FIG. 46, each post 80 includes a prosthesis holder 80a that can protrude inwards from the respective post. The respective holder 80a prevents the medical implant (e.g. prosthesis) 2 from moving axially, radially and rotationally. For example, the respective holder 80a can engage into a cell of the scaffold. The edges of the respective holder 80a are preferably rounded so that no sharp corners can damage the scaffold 20 or the tissue of the leaflets 23.

Alternatively, as shown in FIG. 47, the respective holder 80a can also be configured to engage with a node of the scaffold instead of a cell. Generally, the prosthesis holders 80a are shaped to limit movement of the medical implant (e.g. prosthesis) 2.

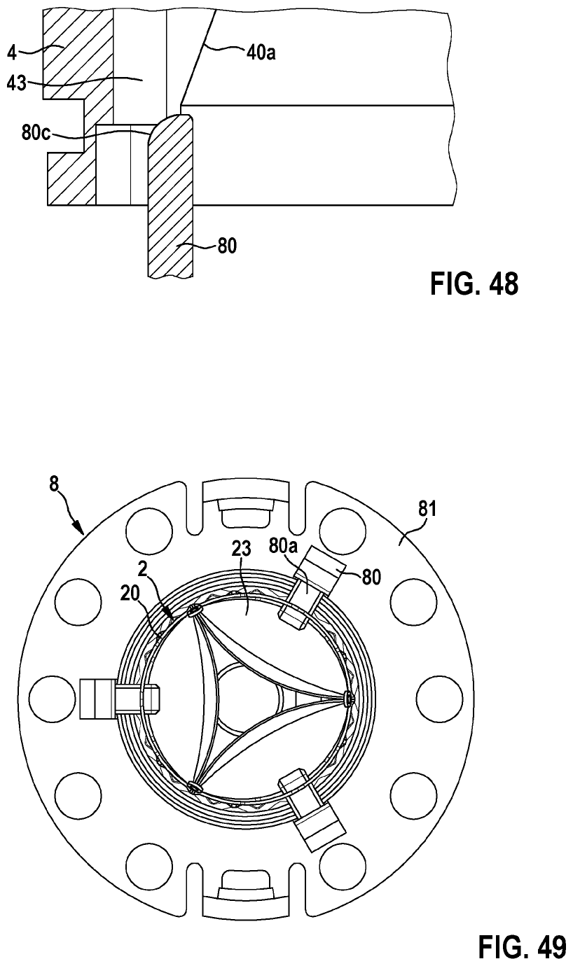

Further, as indicated in FIG. 48, the respective post 80 preferably includes an end with a rounded edge 80c. The rounded edge 80c at the top part of the respective carrier post 80 helps guide and center the post 80 when inserting it into the respective slot 43 of the loading cone 4. According to an alternative concept, the medical implant (e.g. prosthesis) 2 could be fixed to the carrier 8 with sutures and then placed in the cone 4 without posts or guide rails (only the carrier base plate 81 with the scaffold 20 sewn to it). This would allow rotation of the medical implant (e.g. prosthesis) 2 within the loading cone 4. The sutures would have to be cut off before mounting of the implant 2. Magnetic fixation of the medical implant (e.g. prosthesis) 2 is possible, too. Further, the radial force of the scaffold 20 could be used to clamp the medical implant (e.g. prosthesis) 2 into a ring. Furthermore, the medical implant (e.g. prosthesis) 2 has to be mounted to the carrier 8. Particularly, the prosthesis post holders 80a prevent a simple mounting of the medical implant (e.g. prosthesis) 2 in the carrier 8. There are several ways the medical implant (e.g. prosthesis) 2 can be mounted to the carrier 8.

As illustrated in FIG. 49, the medical implant (e.g. prosthesis) 2 is mounted to the carrier 8 having a base plate 81. Each post 80 includes a prosthesis holder 80a that can protrude inwards from the respective post. The respective holder 80a prevents the medical implant (e.g. prosthesis) 2 from moving axially, radially and rotationally. For example, the respective holder 80a can engage into a cell of the scaffold. The edges of the respective holder 80a are preferably rounded so that no sharp corners can damage the scaffold 20 or the tissue of the leaflets 23. It is possible to bend the posts 80 outwards and then place the implant 2 between the posts 80 and release the posts 80 back to their original positions so that the holders 80a engage with the scaffold 20 as described above.

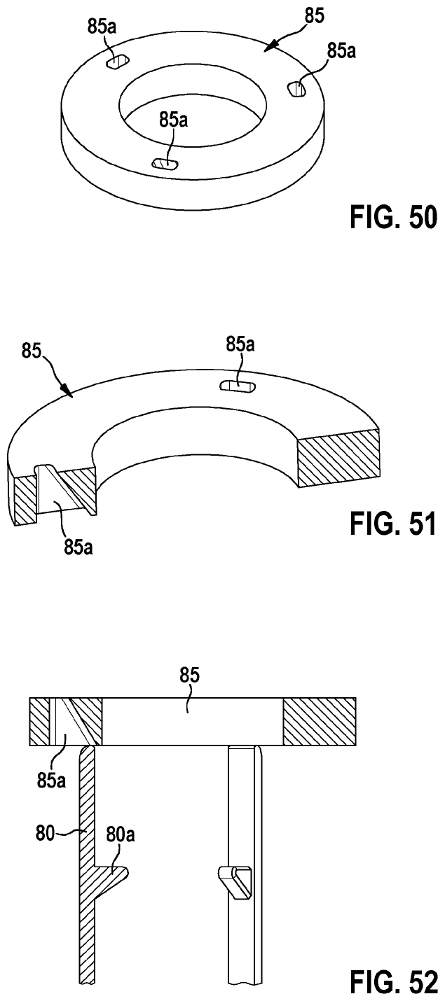

Alternatively, a prosthesis mounting tool 85 can be used as shown in FIGS. 50 to 52 to apply a controlled bending to the posts 80. Such a tool 85 can include a ring with a slot 85a for each post 80. The posts 80 are then spread by a sloped surface of the respective slot 85a when pushing the ring downwards on the posts 80.

According to yet another alternative, the medical implant (e.g. prosthesis) 2 can be arranged on the base plate 81 before placing the posts 80 on the base plate 81. Furthermore, the medical implant (e.g. prosthesis) 2 could be placed on the base plate 81 and the posts 80 could be arranged in place afterwards. This can mean that the posts 80 are bent during mounting or mounted at an angle and then tilted upwards. Similarly, the medical implant (e.g. prosthesis) 2 could be placed after mounting one or two posts 80 wherein the remaining posts are mounted after placement of the implant 2. The medical implant (e.g. prosthesis) 2 could also be placed by spreading the posts 80 by hand.

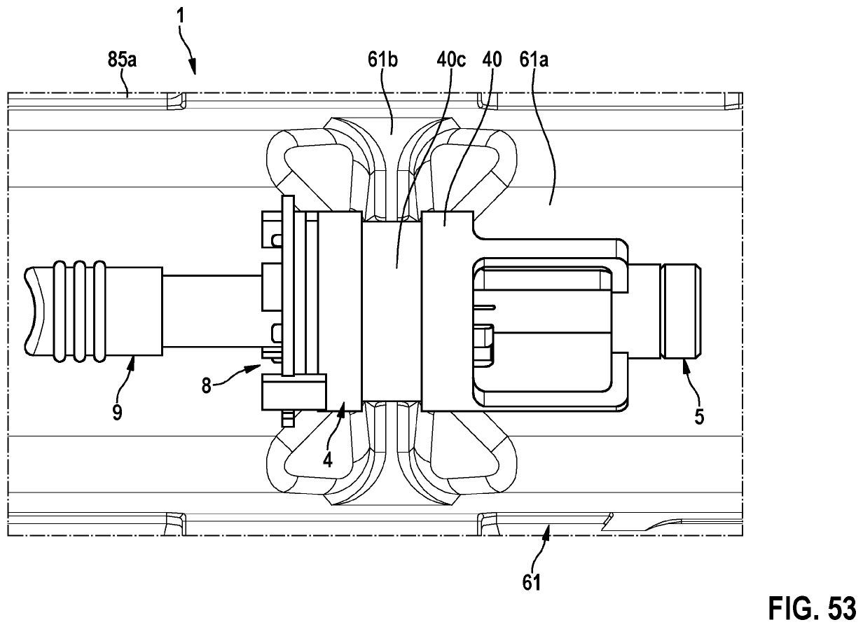

Furthermore, as shown in FIG. 53 (cf. also FIG. 19), the body 40 of the loading cone 4 can include a further circumferential recess (e.g. groove) 40c for fixing the loading cone 4 in the loading basin 61a of the first packaging part 61. Particularly, the loading basin 61a can include a constriction 61b that engages with the recess 40c. Advantageously. this prevents axial movement of the (loading) system 1 during the loading process. Additionally, rotational movement can be prevented, e.g. by using a notch and protrusion or a non-spherical shape, if the connector 33 can be pre-aligned with the (loading) system 1 (and with that the prosthesis connecting elements or eyelets 22).

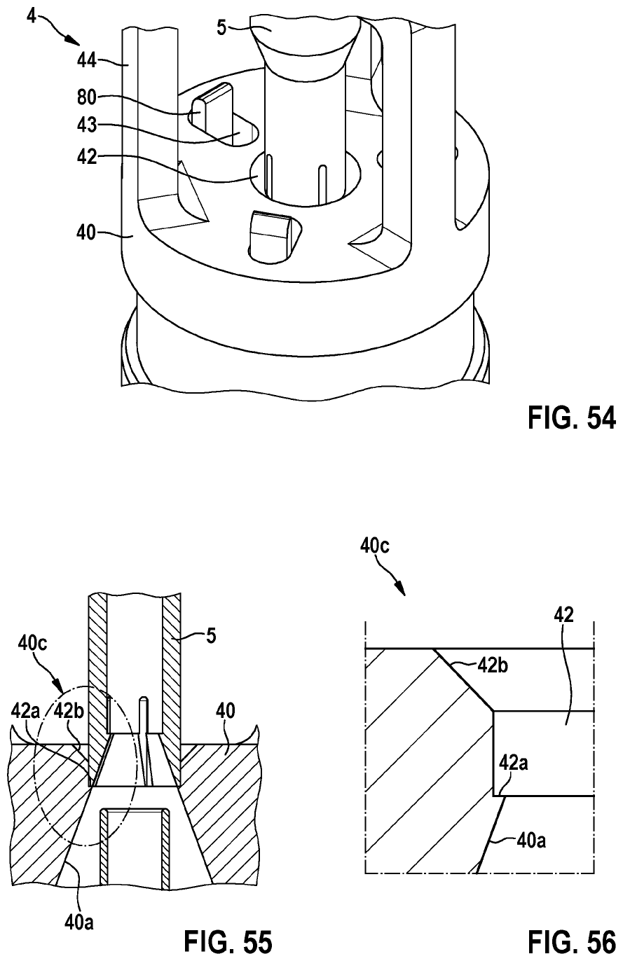

Furthermore, as shown in FIGS. 54 to 56, the loading cone 4 is preferably specifically design to allow a precise guiding and operation of the loading tube 5. For this, on the side of the small diameter, i.e. on the side of the proximal opening 42, the cone 4 includes a circumferential ledge 42a which acts as a hard stop for the inserted loading tube 5. The ledge 42a also allows for a smooth transition between the loading tube 4 and the loading cone's conical inner surface 40a shape during mounting. The cylindrical part of the proximal opening 42 stabilizes the loading tube 5 radially, thus preventing it from expanding. The countersink 42b of the opening 42 centers the loading tube 5 when it is inserted into the proximal opening 42. In order to properly guide the loading tube 5, the cone 4 includes a loading tube guide 45 that is configured to guide the loading tube 5 to the countersunk proximal opening 42 of the body 40 of the loading cone 4. The guide 45 itself may be also countersunk to facilitate insertion of the loading tube 5. The guide 45 is an annular member 45 that includes a central guiding opening 45a and is connected to the body 40 of the cone 4 via (three) spacer bridges 44. The length of the cylindrical part of the loading tube guide 45 defines the alignment of the loading tube 5 with the loading cone 4 (if the cylindrical part is longer, alignment of the loading tube 5 with the cone 4 is better). The spacer bridges 44 create an empty space between the loading tube guide 45 and the body 40 of the loading cone 4. This space is needed to connect the connecting elements 22 (e.g. eyelets) of the medical implant (e.g. prosthesis) 2 to the connector 33 of the delivery catheter 3.

According to an alternative embodiment, the posts 80 can be omitted and the three slots 43 in the body 40 of the loading cone 4 could be removed and the prosthesis carrier 8 would be fully rotatable in the loading cone 4. Furthermore, in case of a solution without an expandable loading tube 5 the cylindrical area 45 and the ledge 42a at the outflow side of the cone 4 could be potentially omitted. Furthermore, different shapes and numbers of spacer bridges 44 are possible to improve visibility of the connector 33. Furthermore, a solution without spacer bridges 44 and loading tube guide 45 could be feasible, where the loading tube 5 inserts directly into the loading cone 4. Furthermore, different angles of the inner surface 40a of the loading cone 4 are possible. The cone 4 does not necessarily have to have a conical inner surface 40a (decreasing uniformly in diameter). A trumpet shape or other tapering shapes would also be feasible.

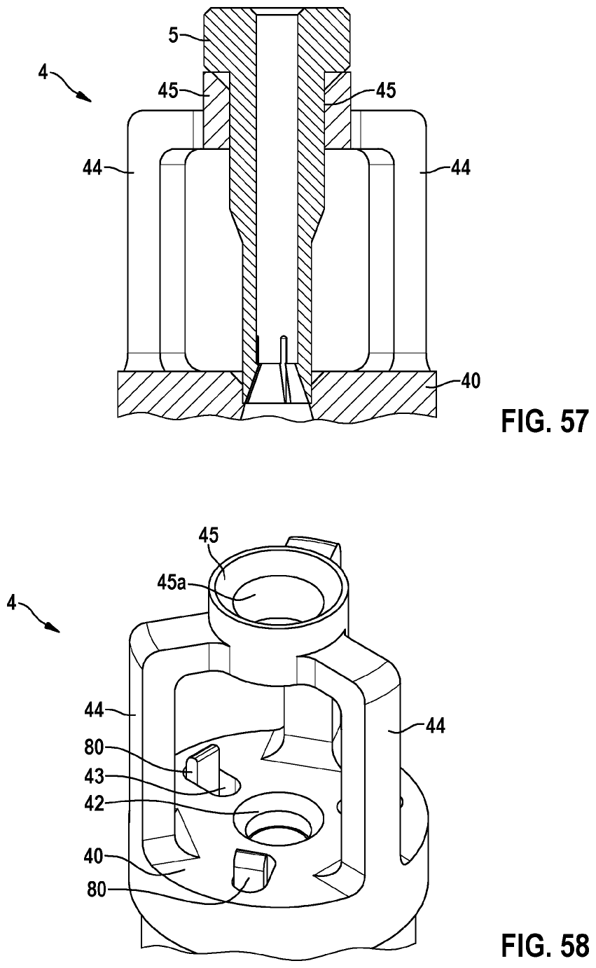

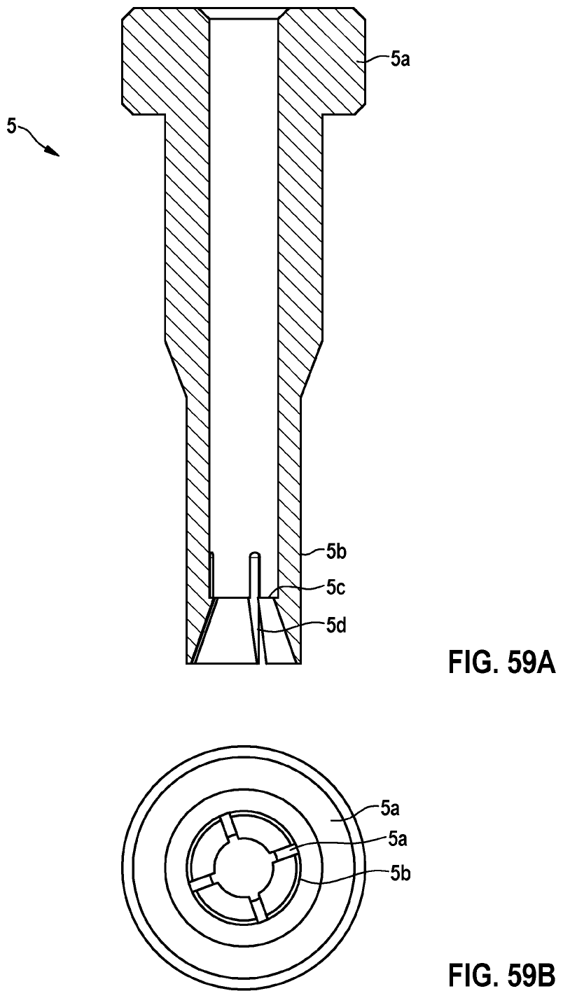

Furthermore, FIGS. 58, 59a and 59b show a preferred embodiment of a loading tube 5 of the system 1 according to the present invention. The main purpose of the loading tube 5 is to support the distal end of the capsule 226 of the delivery catheter 3. The inner diameter of the tube 5 prevents the capsule 226 from flaring and gives it radial support. The end portion 5b of the loading tube 5 preferably contains two major elements. A ledge 5c, which stops the capsule 226 from traveling into the loading cone 4 and (four) radial slots 5d, which allow the end of the tube 5 to spread and the nose cone 220 to pass through the loading tube 5. When the loading tube is fully inserted into the proximal opening 42 of the loading cone 4, this radial expansion is limited by the proximal opening 42 in the loading cone 4, so that the nose cone 220 can only pass through the loading tube 5 if the loading tube 5 is not fully inserted into the loading cone 4 cone. The head 5a of the tube 5 on the opposite side of the end portion 5b of the loading tube 5 facilitates the handling of the tube 5 and could also act as a hard stop that interacts with the guide 45, so that the tube 5 does not extend into the loading cone 4.

According to an alternative configuration, the loading tube 5 could be attached to the loading cone 4 in the mounting configuration by screwing, bayonet, magnetic, wedge, press fit or snapping mechanism. Furthermore, alternatively, the axial fixation of the capsule 226 (which prevents the capsule 226 from diving into the loading cone 4 and the distal end from deforming) with the ledge 5c and the radially expandable end 5b of the loading tube 5 can be replaced by a fixation of the outer sheath or capsule on another level. For example, the outer sheath 202 could axially fixed in the packaging 6. This would also prevent the capsule 226 from diving into the loading cone 4. Furthermore, an additional step could be added, where the medical implant (e.g. prosthesis) 2 is first pulled into the loading tube 5 and then encapsulated within the loading tube 5.

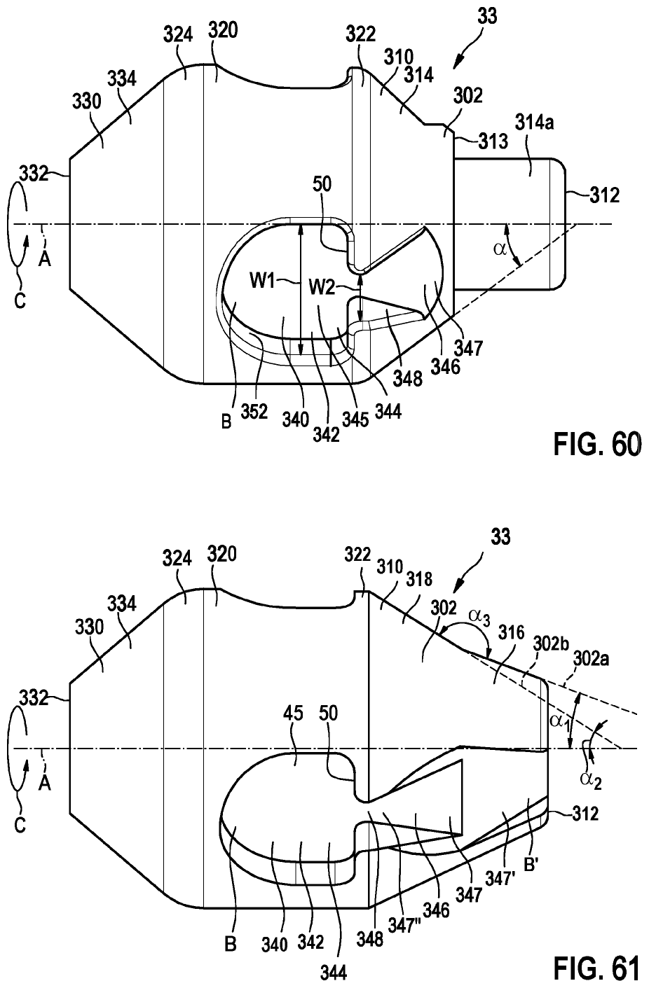

Furthermore, FIGS. 60 and 61 each show an embodiment of a sloped connector 33 according to the present invention. Such a connector 33 allows the connecting elements 22 to slide up to the recesses 340 of the connector 33 so that a splay tube device 9 can be omitted. The connector 33 extends along a central axis A and includes an outer surface 302. Furthermore, as shown in FIG. 60, the connector 33 includes a distal portion 310, a central portion 320 and a proximal portion 330. Particularly, the distal and proximal portions 310, 320 are connected through the central portion 320. Particularly, the central portion 320 can be connected to the distal portion 310 via an intermediate portion 322 providing a transition, particularly a rounded transition, between the central and distal portions 320, 310. The central portion 320 can be connected to the proximal portion 330 through a further intermediate portion 324 that provides a transition, particularly a rounded transition, between the central 320 portion and the proximal portion 330 of the connector 33. According to the invention, the distal portion 310 includes a distal end 312 that faces away from the central portion 320, wherein the distal portion 310 tapers towards the distal end 312. In other words, starting from the distal end 312, the distal portion 310 gets wider (i.e. broadens) towards the central portion 320. Perpendicular to the central axis A, the distal portion 310 can have a circular cross-sectional contour. Preferably, the distal portion 310 includes a conical section 314 so that the distal portion 310 tapers towards the distal end 312. Particularly, the central portion 320 can have a circular cross-sectional contour, too, perpendicular to the central axis A. Furthermore, the central portion 320 can have a cylindrical shape (apart from recesses formed therein). Furthermore, the proximal portion 330 includes a proximal end 332. Particularly, the proximal end 332 faces away from the central portion 320 and the distal end 312. Preferably, the proximal portion 330 tapers towards the proximal end 332. Further, perpendicular to the central axis A, the proximal portion 330 can have a circular cross-sectional contour. Preferably, the proximal portion 330 includes a proximal conical section 334. The proximal conical section 334 tapers towards the proximal end 332. Furthermore, as shown in FIG. 60, for connecting the medical implant (e.g. prosthesis) 2 to the connector 33, the latter includes at least one recess 340, particularly a plurality of recesses 340 such as e.g. two or three recesses 340. In the following the invention will be described in an exemplary fashion with respect to such multiple recesses 340. The respective recess 340 is preferably formed in the central portion 320 and in the distal portion 310 of the connector 33. Particularly, as shown in FIG. 60, the respective recess 340 is formed in the central portion 320, particularly also in the intermediate portion 322 (if present), and in the conical section 314 of the distal portion 310 of the connector 33. I.e. the respective recess 340 extends from the central portion 320 into the distal portion 310. Particularly, the respective recess 340 is delimited by a lateral wall 352 and includes a bottom 342. Preferably, as shown in FIG. 60, the respective recess 340 includes a pocket 344 that is preferably formed in the central portion 320 of the connector 33, and a groove 346 that is preferably formed in the distal portion 310 of the connector 33. Furthermore, the groove 346 is connected to the pocket 344 via a narrow section 348 that can be formed in the intermediate portion 322 (if present). However, generally, the narrow section 348 may also be arranged in the central portion 320 and/or in the distal portion 310. Particularly, the pocket 344 includes a bottom 345 and the groove 346 include a bottom 347, which bottoms 345, 347 form sections of the bottom 342 of the recess 340. Due to the narrow section 348, the pocket 344 includes a width W1 in the circumferential direction C of the connector 1 that is larger than a width W2 of the narrow section 348 in said circumferential direction C so that the pocket 344 forms an undercut that can be engaged by an associated connecting element 22 of the medical implant (e.g. prosthesis) 2/scaffold 20.

According to FIG. 60 the distal portion 310 of the connector 33 includes a conical section 314. Preferably, the conical section 314 include an outer side that includes a particular slope relative to the central axis A, wherein particularly the conical section 314 includes a generatrix 302a that encloses an acute angle α with the central axis A. Particularly, the acute angle α is half the aperture angle of a truncated cone associated to the conical section 314. Furthermore, the bottom 342 of the recess 340 preferably extends in a bottom plane B. In particular, the bottom 347 of the groove 346 and the bottom 345 of the pocket 344 extend in this common bottom plane B as shown in FIG. 61. Furthermore, the distal portion 310 can include a circumferential step 313, wherein the distal end can be formed by a projection 314a adjoining the conical section 314. The projection 314a can be cylindrical with a rounded edge at the distal end 312.

FIG. 61, shows a modification of the embodiment shown in FIG. 60, wherein in contrast to FIG. 60, the distal portion 310 includes a conical first section 316 and a conical second section 318, wherein first section 316 adjoins the second section 318, and wherein the first section 316 forms the distal end 312 of the connector 33. As shown in FIG. 61, the first section 316 include an outer surface having a particular slope relative to the central axis A, which slope differs from a slope of the second section 318, wherein particularly the slope of the second section 318 is steeper than the slope of the first section 316. Particularly, the first section 316 includes a generatrix 302a that encloses an acute first angle α1 with the central axis A. The first angle α1 may be between 20° and 22°, preferably 21°. Likewise, the second section 318 includes a generatrix 302b that encloses an acute second angle α2 with the central axis A that is larger than the first angle α1. The second angle α2 may be between 31° and 33°, preferably 32°. Particularly, the two generatrixes 302a, 302b enclose an obtuse angle α3 as indicated in FIG. 61. Also here, the respective recess 340 can be formed in the central portion 320, particularly also in the intermediate portion 322 (if present), and in the distal portion 310, i.e. the respective recess 340 extends from the central portion 320 into the distal portion 310. Preferably, the groove 346 is formed in the first section 316 and in the second section 318 of the distal portion 310 of the connector as indicated in FIG. 61. Furthermore, the bottom 347 of the groove 346 includes a first groove bottom section 347′ arranged in the first section 316 of the distal portion 310 and a second groove bottom section 347″ arranged in the second section 318 of the distal portion 310, wherein the first groove bottom section 347′ includes a slope with respect to the central axis A that is larger than a slope of the second groove bottom section 347″. Furthermore, the second groove bottom section 347″ and the bottom 345 of the pocket 344 extend in a common bottom plane B, whereas—due to the different slopes—the first groove bottom section 347′ extends in a further bottom plane B′ that extends at an angle with respect to the other plane B.

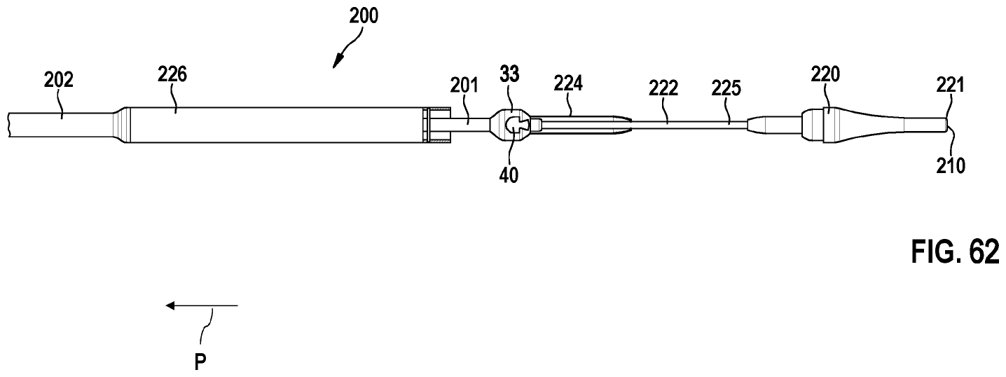

Furthermore, FIG. 62 shows a detail of a delivery catheter 3 according to the present invention that is preferably equipped with the connector 33 according to the present invention. The delivery catheter 3 is configured to deliver the medical implant 2 to an implantation site (preferably via TAVI), wherein the delivery catheter 3 includes a capsule 226 for covering the medical implant 2, which capsule 226 is connected to an outer sheath 202 of the delivery catheter 3 and can be moved in a proximal direction P with respect to an inner sheath 201 of the delivery catheter 3 so that the capsule 226 moves away from a connector 33 of the delivery catheter 3. The connector 33 is connected to a distal end of the inner sheath 201 and is preferably connected via a guidewire tube 225 to a nose cone 220 at a distal end of the delivery catheter 3. When the capsule 226 is closed, it covers the connector 33 and extends up to the nose cone 220 to completely cover the medical implant 2 that then rests on a support element 224 connected to the connector 33.

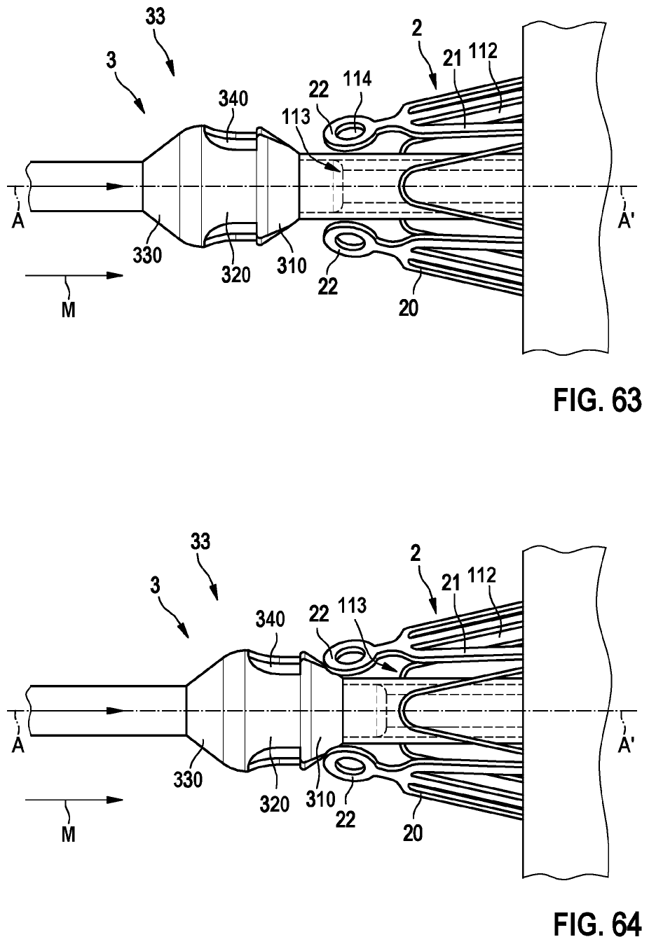

Furthermore, FIGS. 63 to 66 show the process of connecting the connecting elements 22 of a prosthesis 2 to the connector 33 of the delivery catheter 3. The connector 33 includes recesses 340 for receiving the connecting elements 22, which—when the capsule 226 no longer covers the connector 33—move out of the recesses 340 so that the scaffold 20 is released and the medical implant 2 fully expands from a crimped state to an expanded state. Particularly, the delivery catheter 3 can include a catheter tip 220, e.g. in form of a nose cone 220, which forms a distal end 210 of the delivery catheter 3. The delivery catheter 3 further includes an inner 201 sheath including a guidewire lumen 222 and an outer sheath 202 surrounding the inner sheath 201, wherein the outer sheath 202 is connected to a capsule 226 at a distal and of the outer sheath 202. The capsule 226 is configured to cover the medical implant (e.g. prosthesis) 2 when the latter is connected to the connector 33. Further, the connector 33 is connected to a distal end portion of the inner sheath 201 as well as to the nose cone 220 via a guidewire tubing 225. Further, a prosthesis support 224 is connected to the distal end 12 of the connector 1 for supporting the prosthesis 100. The guidewire lumen 222 extends from the inner sheath 201 through the connector 1, the prosthesis support 224 and the guide wire tubing 225 to the catheter tip 220, which includes an outlet 221 for a guide wire that can be inserted into the guidewire lumen 222 to guide the delivery catheter 3 to the implantation site. As in FIG. 60 the distal portion 310 can include a circumferential step (not shown here). Particularly, the medical implant/medical implant (e.g. prosthesis) 2 can be an aortic heart valve medical implant (e.g. prosthesis) 2 that includes a self-expandable tubular scaffold 20 (cf. e.g. FIG. 4) that extends along a central axis A′ and includes a plurality of interconnected struts 21 that form a tubular cell structure surrounding an interior space 112 of the scaffold 20 in which interior space 112 a valve member is arranged and connected to the scaffold 20, which valve member can be formed out of a biological tissue. The scaffold 20 includes a proximal axial opening 113 extending perpendicular to the central axis A′, through which opening 113 blood that passed the valve member can flow into the aorta when the medical implant (e.g. prosthesis) 2 is implanted. For connecting the medical implant (e.g. prosthesis) 2 to the connector 33, the medical implant (e.g. prosthesis) 2 includes connecting elements 22 that can be formed as eyelets including a central opening 114, respectively. The respective connecting element 22 is connected to at least one strut 21 of the stent 20 at a proximal end of the stent 20, wherein the axial proximal opening 113 is arranged between the connecting elements 22 at the proximal end of the stent 20. The capsule 226 can be removed from the medical implant (e.g. prosthesis) 2 when the medical implant (e.g. prosthesis) 2 is connected to the connector 33 via the connecting elements 22 by sliding the outer sheath 202 in a proximal direction P with respect to the inner sheath 201 until the capsule 226 no longer covers the connecting elements 22 residing in the recesses 340 of the connector 33. Such a position of the capsule 226 is shown in FIG. 62. Due to the self-expanding property of the scaffold 20 of the medical implant (e.g. prosthesis) 2, the connecting elements 22 then move out of the recesses 340 and the medical implant (e.g. prosthesis) 2 is completely released from the connector 33. However, as long as the capsule 226 still covers the connecting elements 22, the capsule 226 can be moved over the medical implant (e.g. prosthesis) 2 again by moving the outer sheath 202 and therewith the capsule 226 in the opposite distal direction with respect to the inner sheath 201 thus re-sheathing the medical implant (e.g. prosthesis) 2.

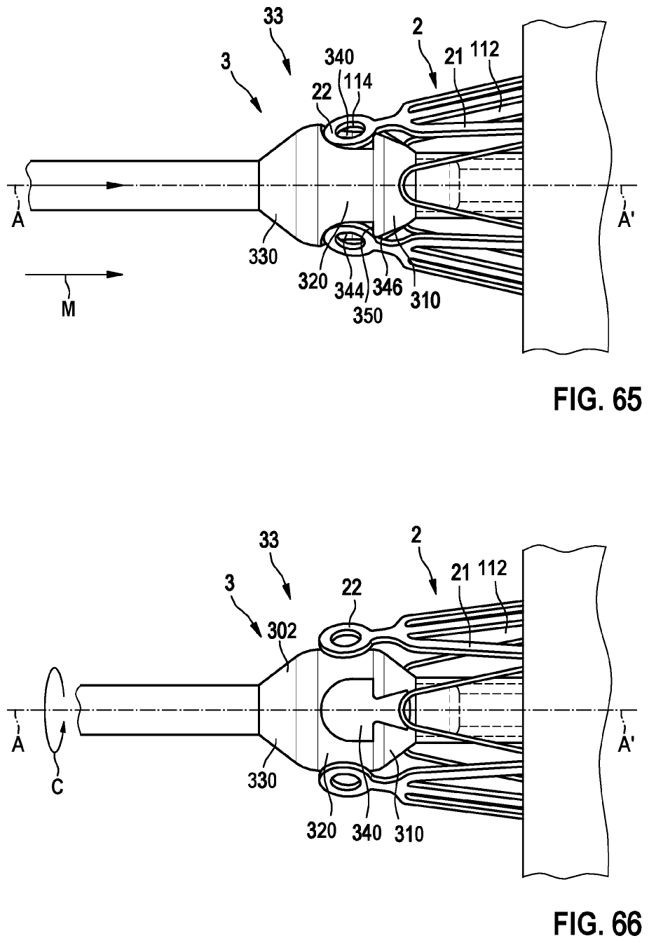

FIG. 63 shows in conjunction with FIG. 64 an embodiment of a method for connecting the medical implant (e.g. prosthesis) 2 to the connector 33 of a delivery catheter 3, wherein the self-expandable medical implant (e.g. prosthesis) 2 is in its collapsed state. FIG. 64 shows the delivery catheter of FIG. 63 and the respective prosthesis with spread connecting elements. Particularly, initially, the connector 33 is positioned such with respect to the medical implant (e.g. prosthesis) 2 that the central axis A of the connector 33 is aligned with the central axis A′ of the scaffold/stent 20 of the medical implant (e.g. prosthesis) 2. The connector 33 is then inserted in an insertion direction M in the proximal axial opening 113 of the medical implant (e.g. prosthesis) 2 (FIG. 64). Thereby, the connector 33, particularly the distal portion 310, spreads the connecting elements 22 apart. Upon further insertion of the connector 33 into the opening 113 of the stent 20, each connecting element 22 engages with a recess 340 of the connector 33 and engages behind the undercut 350 of the pocket 344 of the respective recess 340, which is shown in FIG. 65. This connects the medical implant (e.g. prosthesis) 2 to the connector 33. In this state, the struts 21 connected to the respective connecting element 22 are arranged in the groove 346 of the respective recess 340. The connecting elements 22 can be maintained in this position by the capsule 226 as described above. In case the connecting elements 22 miss the associated recesses 340 upon insertion of the connector 33 into the opening 113 of the stent 20 as shown in FIG. 66, it is possible to rotate the connector 33 and/or the medical implant (e.g. prosthesis) 2 along the circumferential direction C to cause the connecting elements 22 to engage with the recesses 340.

FIG. 65 shows the delivery catheter of FIG. 4 and the respective connecting element connected to the connector of the delivery catheter, wherein the connecting elements are engaged with associated recesses of the connector.

FIG. 66 shows a portion of a prosthesis upon engaging the connecting elements of the prosthesis with the recesses of the connector, wherein the connecting elements are arranged offset with respect to the recesses in a circumferential direction of the connector.

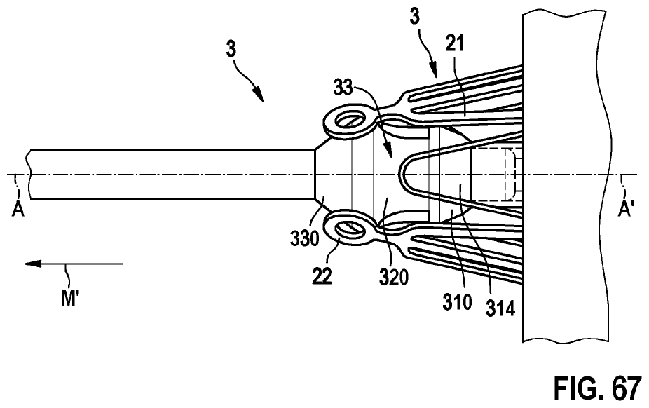

FIG. 67 shows a prosthesis upon engaging the connecting elements of the prosthesis with the recesses of the connector, wherein the connecting elements have been moved passed the recesses of the prosthesis device. In case the connecting elements 22 have been moved past the recesses 340 it is possible to align the connecting elements 22 with the recesses 340 and to move the connector 33 in the opposite direction M′, wherein now the proximal portion 330 spreads the connecting elements 22 apart before they eventually engage with the recesses 340.

The present invention offers the advantages according to which the delivery catheter can be loaded in a safe manner by a single person. No additional packaging is required. The (loading) system is stored within the packaging 6 of the medical implant (e.g. prosthesis) 2 and the delivery catheter 3. Furthermore, less tools are needed for the loading procedure (only a syringe for flushing of the respective lumen is needed). Furthermore, the mounting system 1 protects the distal end of the delivery catheter 3.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A connector for connecting a medical implant to a delivery catheter comprising:
a proximal portion and a distal portion connected to the proximal portion via a central portion, wherein the proximal, distal and central portions extend along a central axis (A);
a distal end of the distal portion facing away from the central portion;
at least one recess formed in the central portion and in the distal portion, wherein the at least one recess is configured to receive a connecting element of the medical implant configured to connect the medical implant to the connector, wherein the distal portion tapers towards the distal end.

2. The connector according to claim 1, wherein the proximal portion comprises a proximal end facing away from the central portion, wherein the proximal portion tapers towards the proximal end.

3. The connector according to claim 1, wherein the distal portion comprises a conical section, wherein an outer surface of the conical section comprises a generatrix that encloses an acute angle (α) with the central axis (A), wherein the acute angle (α) is in the range of between 20° and 60°.

4. The connector according to claim 1, wherein the at least one recess comprises a groove and a pocket connected to the groove via a narrow section of the at least one recess, wherein the groove is formed in the distal portion and the pocket is formed in the central portion.

5. The connector according to claim 4, wherein the groove tapers towards the pocket.

6. The connector according to claim 4, wherein the narrow section comprises a width (W2) extending in a circumferential direction (C) of the connector, and the pocket comprises a pocket width (W1) extending in the circumferential direction (C) of the connector, wherein the width (W2) is smaller than the pocket width (W1).

7. The connector according to claim 4, wherein the at least one recess is configured such that the pocket forms an undercut.

8. The connector according to claim 1, wherein the distal portion comprises a distal first section and a proximal second section.

9. The connector according to claim 8, wherein the distal first section adjoins the proximal second section.

10. The connector according to claim 8, wherein the distal first section is a conical section comprising an outer surface and the proximal second section is a conical section comprising an outer surface, wherein the outer surface of the distal first section comprises a generatrix that encloses a first acute angle (α1) with the central axis (A) and the outer surface of the proximal second section comprises a generatrix that encloses a second acute angle (α2) with the central axis (A), wherein the first acute angle (α1) is smaller than the second acute angle (α2).

11. The connector according to claim 10, wherein the generatrixes of the distal first section and of the proximal second section enclose an obtuse angle (α3), wherein the obtuse angle (α3) is larger than 100°.

12. The connector according to claim 8, wherein the groove comprises a groove bottom comprising a first groove bottom section arranged in the first section of the distal portion and a second groove bottom section arranged in the second section of the distal portion, wherein a slope of the first groove bottom section is larger than a slope of the second groove bottom section.

13. The connector according to claim 1, comprising at least two recesses each formed in the central portion and in the distal portion, wherein each recess is configured to receive a connecting element of the medical implant.

14. A delivery catheter comprising a connector according to claim 1 and an inner sheath comprising a distal end portion, wherein the connector is connected to the distal end portion of the inner sheath.

15. (canceled)

16. A system comprising a connector according to claim 1, the system being configured for connecting a medical implant to a delivery catheter configured for delivering the medical implant to an implantation site, wherein the system comprises:
the medical implant, wherein the medical implant comprises a self-expandable or self-expanding scaffold;
a delivery catheter configured to deliver the medical implant to an implantation site, wherein the delivery catheter comprises a capsule for covering the medical implant and at least one connector according claim 1 configured to connect the delivery catheter to the medical implant;
a loading cone configured to arrange a proximal end portion of the scaffold in a crimped state by decreasing an outer diameter of the proximal end portion of the scaffold, wherein the loading cone comprises a body comprising a distal opening configured to receive the scaffold and an opposing proximal opening, wherein the distal opening comprises a diameter that is larger than a diameter of the proximal opening, and wherein the body of the loading cone comprises an inner surface extending from the distal opening to the proximal opening; wherein the inner surface tapers towards the proximal opening; and
a loading tube configured to be inserted into the proximal opening of the loading cone and to enclose the capsule of the delivery catheter;
wherein the loading cone and the loading tube are pre-mounted on the delivery catheter, and wherein the system comprises a packaging enclosing the delivery catheter, the loading cone, and the loading tube pre-mounted on the delivery catheter.

17-34. (canceled)
